(19) United States
(12) Patent Application Publication
(10) Pub. No.: US 2023/0357854 A1
(43) Pub. Date: Nov. 9, 2023

(54) ENHANCED SEQUENCING FOLLOWING RANDOM DNA LIGATION AND REPEAT ELEMENT AMPLIFICATION

(71) Applicant: Dana-Farber Cancer Institute, Inc., Boston, MA (US)

(72) Inventor: Gerassimos Makrigiorgos, Chestnut Hill, MA (US)

(73) Assignee: Dana-Farber Cancer Institute, Inc., Boston, MA (US)

(21) Appl. No.: 18/022,861

(22) PCT Filed: Aug. 23, 2021
(86) PCT No.: PCT/US2021/047153

§ 371 Date: Feb. 23, 2023

Related U.S. Application Data
(60) Provisional application No. 63/069,502, filed on Aug. 24, 2020.

(51) Int. Cl. C12Q 1/6886; C12Q 1/6855; C12Q 1/6869

(52) U.S. Cl. CPC C12Q 1/6886; C12Q 1/6855; C12Q 1/6869; C12Q 2600/156; C12Q 2600/154; C12Q 1/6806

(57) ABSTRACT

Presently described are methods for enriching regions of a genome in a sample using ligation of fragmented genomic nucleic acid and amplification using repeat elements. The methods can be used for a number of applications, including genome-wide homopolymer indel detection, and enable increasing the amount of information obtained from a limited sample of genomic nucleic acid.

### RELATED APPLICATIONS

This application claims priority to U.S. provisional application No. 63/069,502, filed Aug. 24, 2020, the contents of which are incorporated herein by reference in their entirety.

### GOVERNMENT SUPPORT

This invention was made with government support under RO1 CA221874 awarded by the National Institutes of Health (NIH). The government has certain rights in the invention.

### BACKGROUND

There is mounting evidence that genetic alterations identified in circulating-free DNA of tumor origin (ctDNA) can potentially act as a powerful liquid biopsy based diagnostic tool (Diehl et al., Nat Med, 14, 985-990; Thierry et al., Nat Med, 20, 430-435; Newman et al., Nat Med. 20, 548-554; Bettegowda et al., Sci Transl Med, 6, 224ra24; Diehl et al., Proc Natl Acad Sci USA, 102, 16368-16373). Clinical studies indicate the use of ctDNA to complement (Schwaederle et al., Clin Cancer Res, 22, 5497-5505) or replace (Thierry et al., Nat Med, 20, 430-435) tissue biopsies. Rare mutations identified in ctDNA via digital droplet PCR (ddPCR) or massively parallel sequencing (MPS) can lead to changes in clinical practice (Roschewski et al., Lancet Oncol, 16, 541-549). Despite its promise, technical hurdles in extracting clinically useful information from liquid biopsies persist. The limited amount of ctDNA obtained from a standard blood draw and the excess amount of circulating wild-type (WT) DNA are issues that often compromise the diagnostic results. Frequently, the amount of circulating DNA collected from a standard blood draw does not exceed a few nanograms, thus limiting the amount of information that can be obtained for mutation-based diagnostic purposes. While in principle it may be possible to amplify the original material and then test multiple targets from the amplified product, amplification methods such as polymerase chain reaction (PCR) are known to introduce errors via base mis-incorporation, resulting in false positive mutations following sequencing. Importantly, circulating DNA of tumor origin is highly fragmented into DNA segments of average size of 120-200 bp, which complicates molecular analysis. In view of these limitations in the analysis of circulating DNA, the information obtain ed is restricted, thus limiting clinical applicability. When ctDNA is tested via massive parallel sequencing (MPS), it is important to obtain a high level of information without introducing false-positive signals during the sample handling and preparation prior to sequencing.

### SUMMARY

This disclosure provides methods to increase the information obtained from a limited amount of biological sample containing genomic DNA (e.g., circulating DNA from a blood sample). The methods described herein can be used to determine any number of endpoints, including without limitation genome-wide homopolymer indel detection following genome-wide enrichment of portions of the genome rich in poly-adenine microsatellites.

The invention provides methods for enhancing amplification of regions of the genome adjacent to repeat elements, such as Alu-PCR, Line1-element amplification, or inter-simple-segments-PCR (inter-SS-PCR). The methods of the invention are applicable to both intact genomic DNA (e.g., obtained from biopsies) as well as fragmented DNA (e.g., plasma-circulating-DNA, cfDNA, or ctDNA). The extracted DNA is amplified in a way that enables error correction following sequencing, thus reducing introduction of false mutations resulting from polymerase mis-incorporation or sequencing errors. The accurate, high-sensitivity DNA sequencing provided by the methods of the present disclosure is applicable, among other areas, to the extraction of clinically relevant information from circulating DNA obtained from exceedingly small volumes of blood, e.g., blood obtained from a finger-prick (fingerstick) containing just a few microliters of plasma and pico-gram levels of circulating-DNA. This facilitates minimally invasive testing of circulating-DNA which can be done at regular intervals, to intercept clinically relevant changes in circulating-DNA that indicate tumor status or other endpoints of interest.

The invention in one aspect involves applying random ligation to a sample containing fragmented DNA, e.g., such as a sample obtained from circulating blood. FIG. 2 illustrates this concept, that is, the use of random ligation before inter-Alu-PCR to improve the amount of amplifiable genomic DNA. DNA obtained in fragmented form, e.g., from circulating-DNA, is characterized by repeating genomic elements on different individual molecules, thereby preventing amplification of regions of the genome between two repeat elements (as those regions occur in nature) using primers directed to successive repeat elements (or portions thereof) on single DNA molecules. When the DNA is in minute amounts, such as those obtained from a finger-prick (typically of the order of 10-150 picograms DNA), the ability to capture a substantial genomic fraction using amplification between two repeat elements, as shown at the top of FIG. 2, is limited. By applying random DNA fragment ligation as shown in FIG. 2, the DNA fragments unite to form longer concatemers, thereby generating successive repeat elements and enabling amplification between two repeat elements that can capture a major portion of the genome adjacent a repeat element for analysis in a single DNA amplification reaction.

FIG. 3 demonstrates an example where not every fragment of DNA comprises a repeat element that is used for amplification. In this example, concatemers are formed by the joining/ligation of more than two (e.g., three, four, five, six, seven, eight, nine, or ten or more) fragments of DNA, in which the formed concatemer contains more than one repeat element. The resulting concatemer can now be amplified, since it contains at least two repeat elements.

Further, the ligation of any two DNA fragments creates a junction (fusion) position on the resulting ligated DNA molecule that is unique, as it is highly unlikely that copies of the same two fragments will ligate in the same manner anywhere else in the sample. Accordingly, the fusion point provides a Unique Molecular Identifier (UMI, or molecular barcode) characterizing two ligated DNA fragments, which can be used to eliminate errors in sample preparation and sequencing.

In another aspect of the invention, illustrated by FIG. 12, DNA fragments are ligated to an adapter primer. The DNA fragments that include the adapter primer and also include a repeat element can be amplified by primers directed to (a) the adapter primer and (b) the repeat element. In this aspect, regions adjacent to the repeat element likewise can be amplified.

Accordingly, in some aspects, provided herein is a method of enriching regions or portions of a genome in a sample of genomic nucleic acid comprising: providing a sample containing double-stranded fragments of genomic nucleic acid; applying random ligation conditions to the sample to form a plurality of double-stranded concatemers, each double-stranded concatemer having a first repeat element and a second repeat element and each double-stranded concatemer having a first strand and a complementary second strand; and performing DNA amplification using a pair of primers, wherein a first primer of the pair of primers is complementary to a sequence in the first strand within the first repeat element and the second primer of the pair of primers is complementary to a sequence in the second strand within the second repeat element, wherein performing the DNA amplification amplifies nucleic acid between the first and second repeat elements.

In some embodiments, the method further comprises blunt ending the double-stranded fragments of genomic nucleic acid.

In some embodiments, the amplification comprises PCR or isothermal amplification. In some embodiments, the PCR comprises extending the first primer that is annealed to the first strand of the double-stranded concatemer within the first repeat element and the second primer that is annealed to the second strand of the double-stranded concatemer within the second repeat element for a period of time t1 so that the extended primers are 500-600 bp long. In some embodiments, t1 is 5-60 seconds.

In some embodiments, the method further comprises performing whole-genome amplification on the concatemers before performing the amplification. In some embodiments, the method further comprises forming the sample of fragments of genomic nucleic acid from a sample of intact genomic nucleic acid.

In some embodiments, the method further comprises sequencing regions of a genome, the method comprising sequencing the amplified regions between the first and second repeat elements of any one of the preceding claims. In some embodiments, the method further comprises performing single-stranded or double-stranded consensus techniques with unique molecular identifiers (UMI) to identify amplification errors in sequencing data obtained from the amplified regions between the first and second repeat elements on each concatemer, wherein each UMI comprises at least two base pairs of each fragment on either side of a junction between fragments that form the junction.

In some embodiments, applying random ligation conditions results in an increase in amplifiable nucleic acid by at least 2 PCR cycles compared to a method comprising performing amplification without applying random ligation conditions.

In some embodiments, the first repeat element is a tandem repeat or a portion thereof or an interspersed repeat or a portion thereof, and the second repeat element is a tandem repeat or a portion thereof or an interspersed repeat or a portion thereof. In some embodiments, a tandem repeat is a mega satellite or portion thereof, minisatellite or portion thereof, or a microsatellite of portion thereof. In some embodiments, the repeat element is an interspersed repeat that is a Short Interspersed Nuclear Element (SINE) or portion thereof, or a Long Interspersed Nuclear Elements (LINE) or a portion thereof. In some embodiments, the repeat element is a SINE, and the SINE is an Alu element, Alu, or a portion thereof, wherein the portion thereof is a polyA tail. In some embodiments, the first repeat element is different from the second repeat element.

In some embodiments, applying random ligation conditions comprises blunt end ligation, or single-stranded ligation. In some embodiments, applying random ligation comprises creating blunt ends on the fragments; adding a single dA at the 3′ ends of the blunted fragments to create fragments with a 3′ dA overhang on both strands of the fragments; phosphorylating the 5′ ends of the fragments; adding to the sample double-stranded adapters, wherein each adapter is 4-30 bp long and comprises a 3′ dT and phosphorylated 5′ end on both strands of the adapter and a unique molecular identifier (UMI) such that the UMI on each adapter is different from the UMI on any other adapter in the sample; and applying ligating conditions to allow ligation between the fragments with 3′ dA overhangs and the adapters with the 3′ dT overhangs. In some embodiments, each adapter further comprises 1-2 mismatched bp, wherein the mismatched bp are not the outermost base pairs of the adapter.

In some embodiments, the sample of nucleic acid is obtained from a sample of blood comprising less than 1 ng of nucleic acid (or only a few μl). In some embodiments, the method further comprises determining the number of mutations in the amplified regions of genomic nucleic acid, wherein the number of mutations provides an indication of mismatch repair deficiency or total mutation burden. In some embodiments, the method further comprises determining the number of insertions or deletions in homopolymers or heteropolymers in the amplified regions of genomic nucleic acid, wherein the number of insertions or deletions provides an indication of microsatellite instability. In some embodiments, the method further comprises determining the number of copies a gene of interest in the amplified regions of genomic nucleic acid, wherein the number of copies of the gene of interest provides an indication of disease. In some embodiments, the method further comprises determining the number of methylated forms of a gene of interest in the amplified regions of genomic nucleic acid. In some embodiments, the method further comprises determining the number of a short tandem repeat in the amplified regions of genomic nucleic acid and comparing to the number of the short tandem repeat in a reference sample.

In some embodiments, a method of enriching portions of a genome in a sample of genomic nucleic acid comprises providing a sample containing double-stranded fragments of genomic nucleic acid; creating blunt ends on the fragments; adding a single dA at the 3′ ends of the blunted fragments to create fragments with a 3′ dA overhang on both strands of the fragments; phosphorylating the 5′ ends of the fragments; adding to the sample double-stranded adapters, wherein one end of the adapter comprises a first 3′ dT overhang and 5′ phosphorylated end, and the other end of the adapter is blunted or comprises a second 3′ dT overhang and 5′ phosphorylated end, and wherein each adapter comprises a common hybridization sequence and a unique molecular identifier (UMI) that is between the first or second dT overhang and the common hybridization sequence and is different from the UMI on any other adapter in the sample; applying ligation conditions to the sample to form a plurality of double-stranded adapter ligated fragments, each adapter ligated fragment comprising a first strand and a second strand and an adapter on at least one end of the fragment, wherein at least some of the adapter ligated fragments comprise a repeat element; and performing amplification using a pair of primers, wherein a first primer of the pair of primers is complementary to a sequence in the first strand within the repeat element and the second primer of the pair of primers is complementary to a sequence in the second strand within the common hybridization sequence on the adapters, wherein performing the amplification amplifies the nucleic acid between the far ends of the repeat element and the common hybridization sequence on the adapters.

In some embodiments, the concentration of the first primer is at least 5 times higher than the concentration of the second primer. In some embodiments, the amplification comprises PCR or isothermal amplification. In some embodiments, PCR amplification comprises 2-20 cycles of touch-down PCR followed by COLD-PCR and/or step-up PCR to preferentially amplify repeat elements with a deletion. In some embodiments, PCR amplification comprises extending the first primer that is annealed to the first strand of the adapter ligated fragment within the repeat element and the second primer that is annealed to the second strand of the adapter ligated fragment within the common hybridization sequence on the adapter for a period of time t1 so that the extended primers are 500-600 bp long. In some embodiments, t1 is 5-60 seconds.

In some embodiments, adding to the sample double-stranded adapters and applying ligation conditions results in an increase in the number of repeat elements captured by at least 10-fold compared to a method comprising performing amplification without adding to the sample double-stranded adapters and applying ligation conditions.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1 illustrates use of the methods described herein for identification of microsatellite instability (MSI) status and tracing of MSI and/or tumor status in plasma using Alu-PCR-MSI-tracer (SEQ ID NOs: 3-6 (top-bottom)).

FIG. 2 illustrates that random inter-ligation of fragmented DNA enhances Alu-PCR amplification, while also embedding unique molecular identifiers (UMIs) at the junctions between fragments of DNA.

FIG. 3 illustrates that inter-ligation of fragmented DNA enhances Alu-PCR amplification, while also embedding unique molecular identifiers (UMIs) similar to FIG. 2, but with ligation to non-Alu fragments as well.

FIG. 4 provides an example of materials and methods to enable DNA blunting followed by random ligation. Primers used for inter-Alu-PCR were ‘tail Alu primer’ and ‘head Alu primer’.

FIGS. 5A-5B show a demonstration of improved inter-Alu-PCR following random inter-ligation of fragmented genomic DNA. Amplification results are compared for samples containing blunting enzyme mix and ligase (S1 and S2), no blunting enzyme mix (S3), no ligase (S4), no blunting enzyme and no ligase (S5), no template control (NTC) in blunting and ligation (S6), no blunting enzymes and no ligase (incubated on ice) (S7), sheared HMC in water (no buffer control, incubated on ice) (S8).

FIG. 6 shows a comparison of numbers of different Alu sites obtained without ligation, versus Alu sites obtained via ligation, when Alu-PCR is followed by sequencing. Alu sites represented by more than 20 sequencing reads are included.

FIGS. 7A-7C shows examples of somatic poly-A insertions and deletions (indels) detected via sequencing of inter-ligated inter-Alu-derived poly-As. Examples of somatic indels detected on fragmented DNA from tumor tissue (MM14), compared to matched normal tissue DNA from the same patient (MM13) following inter-ALU-PCR and using the scheme described above. Fragmented genomic DNA (1 ng) from an MSI colon CA patient was used as starting material. The target examined was the poly-A tail of ALU elements dispersed among several genomic regions. Inter-ALU-sequence alignment was done using the Burrows-Wheeler Aligner (BWA) algorithm (Harvard School of Public Health (HSPH) core facility).

FIG. 8 shows serial dilution of tumor DNA (CT18) into excess normal DNA (CN18), followed by Alu-PCR and sequencing. The indels from tumor DNA can be detected at ratios as low as 0.01% tumor DNA to normal DNA. A HiSeq Illumina sequencer was used for sequencing, and microsatellites were analyzed using MSI Sensor software and MSI Tracer software. 0.3× downsampling (to select a subset of sequencing reads) was used on the samples Cn18, 0.01% CT18, and 0.03% CT18. No downsampling was used on the rest of the samples.

FIG. 9 shows random inter-ligation of fragmented DNA following A-tailing, using T-tailed DNA adapters.

FIG. 10 shows T-tailed DNA adapters with 8-12 nucleotides and optionally one or more nucleotide mismatches that enables differentiation of top and bottom strands during sequencing.

FIG. 11 shows random inter-ligation of fragmented DNA followed by whole genome amplification, and then by Alu-PCR.

FIG. 12 shows ligation of UMI-containing adapters to fragmented DNA, followed by Alu PCR using Alu elements and a hybridization sequence in the adapters. The adapters have a 3′ dT overhang and a blunt end or two 3′ dT overhangs, a UMI, and a hybridization sequence that can be used for amplification. The adapters are also phosphorylated at the 5′ ends.

FIG. 13 shows a comparison of the number of Alu elements captured by various approaches for inter-Alu-PCR. Results are shown for amplification from intact DNA, fragmented DNA, adapter-ligated DNA using Alu-binding primers, and Adapter-ligated DNA using one Alu-binding primer and on adapter-binding primer.

FIG. 14 shows the results of a clinical study for detection of microsatellite instability (MSI) tumors using plasma-circulating DNA from colon cancer patients analyzed by the approach shown in FIG. 12. Circulating DNA from colon cancer patients with either MSI-positive tumors or MSI-stable (MSS) tumors was interrogated using the approach in FIG. 12. Samples with an MSI-Tracer score exceeding the indicated threshold were classified as MSI-positive. Tumors in late stages (II, III, or IV) were more likely to be classified correctly.

### DETAILED DESCRIPTION

Next-generation, massively-parallel sequencing (MPS) technologies have transformed the landscape of genetics through their ability to produce giga-bases of sequence information in a single run. However, the sequencing cost, computation workload and amount of sample DNA required are still too high for large scale population analysis by means of whole-genome sequencing. There is clearly a need for pre-sequencing capture of subsets of the genome to reduce these requirements.

Repeat element amplification is an alternative avenue that can extract a useful genomic portion in a single DNA amplification reaction from a genomic DNA of interest. For example, Alu-transposons are a family of primate-specific short interspersed nucleotide elements (SINE) of ˜300 bp derived from 7SL RNA (Mei et al., BMC Genom, 12, 564). Although Alu elements were once considered as ‘junk DNA’, their biological importance, and in particular their influence on genome instability, is being increasingly recognized (Konkel et al., Semin Cancer Biol, 20, 211-221). By placing PCR primers on the repeat Alu sequences, it is possible to amplify portions of the genome present between two adjacent Alu sequences (‘inter-Alu PCR’), or portions of the Alu elements themselves (‘intra-Alu-PCR’) (Mei et al., BMC Genom, 12, 564). Since there are more than 1 million Alu elements interspersed in the human genome, Alu-PCR can select a substantial portion of the genome for sequencing following a single PCR reaction, thereby accelerating analysis and reducing cost for sample preparation. Further, in view of the multiplicity of Alu elements, inter-Alu-PCR and intra-Alu-PCR can be performed from minute amounts of starting DNA material, thereby reducing the requirements on starting amount of DNA. The advantages of sequencing DNA ‘captured’ by a single inter-Alu-PCR amplification using intact genomic DNA obtained from biopsies have been described (Mei et al., BMC Genom, 12, 564).

Unfortunately, when Alu-PCR is performed on fragmented DNA, such as circulating DNA obtained from plasma, Alu-PCR is much less efficient because, in view of the highly fragmented nature of DNA, the presence of two Alu elements on the same cfDNA fragment is less common. Thus, while inter-Alu-PCR (or PCR between any two repeat elements) can still be conducted when cfDNA is the starting material, the genomic portion that can be captured for sequencing is limited.

By enabling generation of two or more contiguous repeat elements on single DNA molecules, the methods described in the current disclosure enable a large fraction of DNA to be captured via a single DNA amplification.

### A Method of Enriching Regions of a Genome Using Primers Complementary to Repeat Elements

Provided herein is a method of enriching regions or portions of a genome in a sample of genomic nucleic acid. In some embodiments, such a method comprises providing a sample containing double-stranded fragments of genomic nucleic acid. In some embodiments, a method of enriching regions of genomic nucleic acid comprises applying random ligation conditions to the sample to form a plurality of double-stranded concatemers, each double-stranded concatemer having a first repeat element and a second repeat element and each double-stranded concatemer having a first strand and a complementary second strand; and performing DNA amplification using a pair of primers, wherein a first primer of the pair of primers is complementary to a sequence in the first strand within the first repeat element and the second primer of the pair of primers is complementary to a sequence in the second strand within the second repeat element, wherein performing the DNA amplification amplifies nucleic acid between the first and second repeat elements.

As used herein, enriching regions of a genome means amplifying some regions of the genome relative to other regions. In some embodiments, the amplified region is enriched relative to other regions of the genome by at least 2-fold (e.g., by at least 2-fold, at least 5-fold, at least 10-fold, at least 50-fold, at least 100-fold, at least 200-fold, at least 500-fold, at least 1000-fold, at least 2000-fold, or at least 5000-fold).

### Samples of Genomic DNA

In some embodiments, a sample of genomic DNA comprises fragmented DNA (e.g., as that found in circulating cell-free samples of DNA). In some embodiments, fragments of nucleic acid are 20-5000 bp long (e.g., 20-100, 20-150, 50-150, 50-200, 100-500, 100-1000, 100-2000, 500-2000, or 1000-5000 bp long). In some embodiments, a fragment of nucleic acid is 120-200 bp long. In some embodiments, the average length of fragments in a sample of nucleic acid is 120-200 bp long. In some embodiments, a sample containing fragments of nucleic acid will comprise at least some (e.g., 0.0001% or more, 0.001% or more, 0.01% or more, 0.1% or more, 1% of more, 2% or more, 5% or more, 10% or more, or 20% or more) fragments that contain at least one repeat element.

In some embodiments, a sample of nucleic acid for use in any one of the methods disclosed herein contains DNA that is intact. As used herein, with reference to DNA or nucleic acid, “intact” means DNA or nucleic acid that is greater than 5000 bp long and has not been subjected to fragmenting. In some embodiments, the methods comprise forming fragments of nucleic acid from a sample of intact nucleic acid. The fraction of total repeat elements (e.g., Alu elements) that can be amplified following the methods described herein may exceed the repeat elements (e.g., Alu elements) captured in amplification methods using intact genomic DNA. In intact genomic DNA there are successive repeat elements (e.g., Alu elements) (meaning next to each other without an additional repeat element of the same kind intervening) that are far apart from each other (e.g., several kb), and, because they are far apart, cannot be amplified effectively by methods such as inter-Alu-PCR. In contrast, the DNA from plasma is randomly fragmented into pieces typically <200 bp, and these small pieces typically contain only a single repeat element. As described herein, these repeat elements can be brought close to one another via random-inter-ligation, bringing them close enough to permit effective amplification. Accordingly, in some embodiments, a sample of intact nucleic acid is fragmented to form fragments of nucleic acid to which random ligation and an amplification method using one or more repeat elements is applied. Methods of fragmenting nucleic acids are known in the art. Non-limiting examples of fragmenting nucleic acid include using an enzyme such as a Shearase enzyme, and using mechanical or acoustic forces (e.g., see US20180298425).

In some embodiments, genomic nucleic acid is isolated from a biological sample (e.g., blood, urine, cerebrospinal fluid, or tissue, e.g., a tumor tissue). In some embodiments, the biological sample is obtained from a mammal (e.g., a human). In some embodiments, a biological sample is obtained from a mammal but is comprised of foreign genome, e.g., a viral genome. In some embodiments, a sample of genomic nucleic acid is DNA (e.g., that of a human). In some embodiments, a sample of genomic nucleic acid is RNA (e.g., that of an RNA virus). In some embodiments, the nucleic acid sample is obtained from a sample of blood that is only a few μl in volume (e.g., less than 200, less than 100, less than 50, less than 20, less than 10, less than 5, or less than 2 μl in volume) and/or comprising less than 1 ng of nucleic acid (e.g., less than 1 ng, less than 500 pg, less than 200 pg, less than 100 pg, less than 50 pg, or less than 20 pg of nucleic acid).

### Concatemers and Repeat Elements

A concatemer is a continuous nucleic acid molecule made up of at least two repeat elements, i.e., a first repeat element and at least a second repeat element. In some embodiments, a concatemer is formed following random ligation of fragmented nucleic acid in a nucleic acid sample. A concatemer may be double-stranded or single-stranded. In some embodiments, a concatemer is 100-5000 bp long (e.g., 100-5000 bp, 200-5000 bp, 500-5000 bp, 500-1000 bp, 1000-5000 bp, 1000-3000 bp, or 4000-5000 bp). In some embodiments, a concatemer is at least 10 bp long (e.g., at least 10, at least 50, at least 100, at least 150, at least 200, at least 500, at least 1000, at least 2000, or at least 5000 bp long). It is to be understood that random ligation will result in varying lengths of concatemers in a sample. For example, some of all concatemers formed in a sample following random ligation may be less than 1000 bp, while other concatemers in the sample may be 3000-5000 bp long. In any of the embodiments in this application, when length is indicated, the length can be absolute or average length in a sample.

As used herein, a repeat element is a nucleic acid sequence of at least 5 nucleotides in length that appears in at least 50 copies across a genome. In some embodiments, the repeat element is at least 10 bp long and occurs at least 100, at least 500, or at least 1000 times in the genome. In some embodiments, repeat elements are used to amplify portions or regions of the genome with any one of the methods described herein.

A repeat element may be highly repetitive or moderately repetitive. In some embodiments, highly repetitive repeat elements are 5-10 bp long and occur approximately 106 copies per haploid genome. In some embodiments, moderately repetitive repeat elements are 150-5000 bp long and occur approximately 103-105 copies per haploid genome. In some embodiments, a repeat element is the entirety of a highly repetitive or moderately repetitive element. In some embodiments, a repeat element is a portion of a highly repetitive or moderately repetitive element.

An example of a highly repetitive repeat element is satellite DNA. In some embodiments, satellite DNA is represented by monomer sequences, usually less than 2000-bp long, repeated throughout a genome, up to 105 copies per haploid.

Examples of moderately repetitive repeat elements are tandem repeats or interspersed repeats. Tandem repeats can be minisatellites, megasatellites or microsatellites (e.g., dinucleotide repeats). Examples of minisatellites include hypervariable minisatellites, telomeric minisatellites, and subtelomeric minisatellites). Interspersed repeats can be RNA transposons or DNA transposons. RNA transposons can be Long Terminal Repeats (LTRs) or non-LTRs. In some embodiments, an LTR is an Endogenous Retrovirus (ERV). Non-limiting examples of non-LTRs are Long Interspersed Nuclear Elements (LINEs) and Short Interspersed Nuclear Elements (SINEs).

In some embodiments, a repeat element is the entirety of, or a portion of, a satellite DNA. In some embodiments, a repeat element is the entirety of, or a portion of, a tandem repeat. In some embodiments, a repeat element is the entirety of, or a portion of, a minisatellite, megasatellite, or microsatellite. In some embodiments, a repeat element is the entirety of, or a portion of, a hypervariable minisatellite, a telomeric minisatellite, or a subtelomeric minisatellite. In some embodiments, a repeat element is the entirety of, or a portion of, an RNA transposon or a DNA transposon. In some embodiments, a repeat element is the entirety of, or a portion of, an LTR or an ERV. In some embodiments, a repeat element is the entirety of, or a portion of, a LINE or a SINE. In some embodiments, a repeat element is the entirety of, or a portion of, an Alu element.

A microsatellite is a tract of repetitive DNA in which certain DNA motifs (ranging in length from one to six or more base pairs) are repeated, typically 5-50 times. Microsatellites occur at thousands of locations within an organism's genome. They have a higher mutation rate than other areas of DNA, which can be indicative of diseases (e.g., cancer). In some embodiments, microsatellites are also called short tandem repeats (STRs) or simple sequence repeats (SSRs). Microsatellites in a sample of nucleic acid can be of multiple types and of varying length. In some embodiments, minisatellites are of larger length than microsatellites (e.g., up to 100 bp). Non-limiting examples of microsatellites are mono-nucleotide repeats (e.g., AAAAAAAAA), di-nucleotide repeats (e.g., ACACACACACACACA (SEQ ID NO: 1)), or trinucleotide repeats (e.g.,CAGCAGCAGCAGCAGCAG (SEQ ID NO: 2)). In some embodiments, a microsatellite has a repeat of more than three nucleotides. A single sample of DNA can have mono-nucleotide repeats, di-nucleotide repeats, and/or tri-nucleotide repeats, each of varying lengths. For example, a sample of nucleic acid may comprise a poly-A repeat that is of 15 bp, 18 bp, 25 bp, and 40 bp. It may also comprise CAG repeats of multiple lengths. In some embodiments, microsatellites may be telomeres, or portions thereof.

In some embodiments, a microsatellite in wild-type nucleic acids is at least 5 bp or nucleotides long. In some embodiments, a microsatellite is 5-100 bp or nucleotides long. In some embodiments, a microsatellite in wild-type nucleic acids is at least 5 repeats long. In some embodiments, a microsatellite is 5-100 repeats long. For mono-nucleotide repeats, the repeating element is one nucleotide. For dinucleotide repeats, the repeating element is two nucleotides long. For tri-nucleotide repeats, the repeating element is three nucleotides long. Therefore, a microsatellite with di-nucleotide repeats having the same number of repeats as a microsatellite with mono-nuclear repeats will be twice as long as the microsatellite with mono-nuclear repeats.

Interspersed repeats are repeat elements that are dispersed throughout a genome (e.g., not adjacent to one another). In some embodiments, an interspersed repeat is a Short Interspersed Nuclear Element (SINE), or a Long Interspersed Nuclear Element (LINE). In some embodiments, an interspersed repeat is a transposable element. A transposable element is a nucleic acid sequence that can change its position throughout a genome. In some embodiments, a transposable element may be an Alu element.

### Random Ligation

As used herein, ligation is the joining of at least two nucleic acid molecules to form a longer nucleic acid molecule, e.g., through the action of an enzyme (e.g., a ligase). In some embodiments, ligation is random ligation. Random ligation is the joining of at least two nucleic acid molecules within a sample through ligation wherein the identity of the nucleic acid molecules involved in the ligation reaction is not controlled or known beforehand. In some embodiments, the at least two nucleic acid molecules are different sequences.

In some embodiments, ligation conditions comprise blunt-end DNA ligation. Blunt-end DNA ligation is ligation that does not involve base-pairing between overhanging nucleic acids of one nucleic acid molecule to overhanging nucleic acids of another nucleic acid molecule. In some embodiments, double-stranded nucleic acid molecules are ligated to one another. In some embodiments, single-stranded nucleic acid molecules are ligated to one another. In some embodiments, polyethylene glycol (PEG) may be added to the ligation reaction for the purpose of reducing the flexibility of single-stranded DNA, thereby reducing self-circularization. In some embodiments, PEG comprises about 5%, about 6%, about 7%, about 8%, about 9%, about 10%, about 11%, about 12%, about 13%, about 14%, about 15%, about 16%, about 17%, about 18%, about 19%, or about 20% of the ligation reaction solution by volume.

In some embodiments, the fragments of genomic nucleic acid are treated to create blunt ends before being exposed to ligation conditions. In some embodiments, fragments of double-stranded nucleic acid are ligated to form double-stranded concatemers. In some embodiments, fragments of single-stranded nucleic acid are ligated to form single-stranded concatemers.

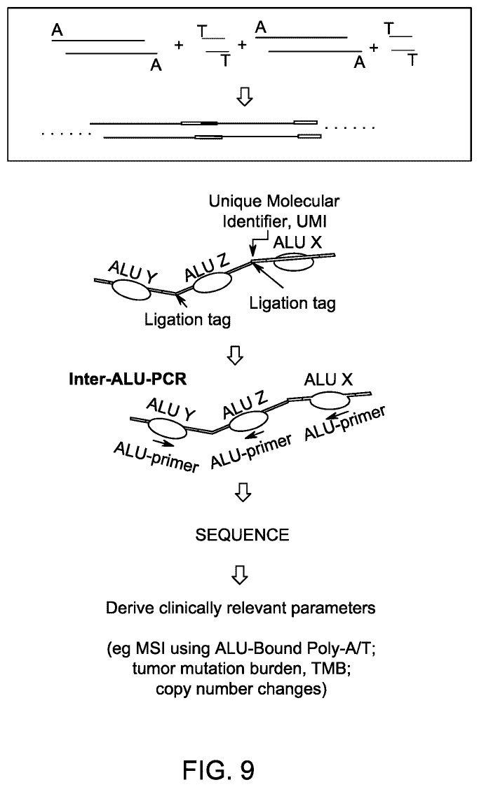

In some embodiments, nucleic acid adapters are added to the ligation reaction. The following provides an example of a ligation method using adapters. DNA fragments in the sample of nucleic acid are treated to create blunt ends, and a single deoxyadenosine (dA) is added at the 3′ ends of the blunted fragments using dA-tailing. Double-stranded DNA adapter tags are then added to the sample that have a 3′ deoxythymidine (dT) on both strands of the adapter and are phosphorylated on the 5′ ends of both strands of the adapter. Ligation conditions as described above are applied to the sample to allow ligation between fragments with dA overhangs and adapters with a dT overhangs, and as a result of the ligation, concatemers of nucleic acid that comprise fragments ligated to DNA adapters are formed (see, e.g., FIG. 9). In some embodiments, the DNA fragments are 5′ phosphorylated.

In some embodiments, the DNA adapters are 4-20 bp long (e.g., 4-20 bp, 5-18 bp, 6-16 bp, 7-14 bp, 8-12 bp, or 9-10 bp long). In some embodiments, each adapter has a unique molecular identifier (UMI) that is different from that on any other adapter in the sample. In some embodiments, the UMI is at least 2 bp long (e.g., at least 2 bp long, at least 3 bp long, at least 4 bp long, at least 5 bp long, at least 6 bp long, at least 7 bp long, at least 9 bp long, or at least 10 bp long). In some embodiments, the adapters comprise a 1 or 2 bp mismatch in the center of the adapter. Such a mismatch can be used to distinguish between the two strands of the adapter, and thus nucleic acid sequence adjacent to the adapter on a concatemer (Genome Res., 27, 491-499).

### Primers

In some embodiments, the methods disclosed herein comprise performing amplification of the concatemers using primers. A primer is a short nucleic acid sequence that provides a starting point for DNA synthesis. In some embodiments, a first primer and a second primer are used. In some embodiments, the primers are complementary to a repeat element on the concatemers, or to a portion of the repeat element. For example, both the first and second primer may be complementary to part of a poly-A tail of an Alu element. In some embodiments, the first primer and second primer are complementary to the entirety of a repeat element on the concatemer, or they are complementary to different repeat elements or parts/portions thereof. For example, a first primer may be complementary to part of the poly-A tail within an Alu element, and a second primer may be complementary to part of a tandem site duplication of an Alu element or part of a telomere. It is to be understood that more than two primers and more than two repeat elements may be used to amplify regions of a genome using any one of the methods described herein. For example, three different PCR primers can be used that each bind to a different repeat element. In some embodiments, one primer is a forward primer and two primers are reverse primers. In some embodiments, two primers are forward primers and one primer is a reverse primer. There is no limitation on the number of primers that can be used in any one of the methods described herein.

### Amplification

In some embodiments, the methods disclosed herein comprise performing amplification to amplify nucleic acid between a first and second repeat element or a repeat element and a common hybridization sequence that is present in an adapter ligated to a fragment of genomic nucleic acid (the latter being an embodiment discussed below).

In some embodiments, amplification is accomplished by PCR. The amplification may be performed using any one of numerous variations of PCR, for example, standard PCR conditions or COLD-PCR conditions. Any variation of COLD-PCR (e.g., temperature independent/tolerant COLD-PCR) can also be performed using primers that are complementary to a repeat element or a common hybridization sequence comprised in an adapter that is ligated to a fragment of genomic nucleic acid. COLD-PCR and its derivatives, e.g., Temperature-Tolerant COLD-PCR are disclosed in the following patent applications: US 2014/0051087, US 2016/0186237, US 2018/0282798, U.S. Pat. No. 8,455,190, US 20160186237.

In some embodiments, the length of time for primer extension in a PCR reaction is used to control the length of the extended primers or amplicons formed by the amplification. In some embodiments, the PCR comprises extending the primers annealed to concatemers so that the resulting amplicons are on average about 100-1000 bp, about 200-900 bp, about 300-800 bp, about 400-700 bp, or about 500-600 bp long. In some embodiments, the extension step of the PCR is performed for about 5 to about 60 seconds (e.g., about 5 seconds, about 10 seconds, about 15 seconds, about 20 seconds, about 25 seconds, about 30 seconds, about 35 seconds, about 40 seconds, about 45 seconds, about 50 seconds, about 55 seconds, or about 60 seconds). In some embodiments, the extension time is 30 seconds.

In some embodiments, DNA amplification is performed by isothermal DNA amplification. Non-limiting examples of isothermal DNA amplification are transcription mediated amplification, nucleic acid sequence-based amplification, strand displacement amplification, rolling circle amplification, loop-mediated isothermal amplification, isothermal multiple displacement amplification, helicase-dependent amplification, single primer isothermal amplification, recombinase polymerase amplification, and circular helicase-dependent amplification (Gill et al., Nucleosides Nucleotides Nucleic Acids, 27, 224-243).

Amplification as used herein refers to amplifying some portions of a molecule or multiple DNA molecules relative to other portions. In some embodiments, the amplified nucleic acid is enriched relative to other DNA in the sample by at least 2-fold, at least 5-fold, at least 10-fold, at least 50-fold, at least 100-fold, at least 200-fold, at least 500-fold, at least 1000-fold, at least 2000-fold, or at least 5000-fold.

In some embodiments, whole-genome amplification (Hosono et al., Genome Res., 13, 954-964) is performed as a step of any one of the methods described herein after ligation of fragments of nucleic acid to one another or fragments of nucleic acid to adapters, but before amplification using repeat elements or a repeat element/s and a common hybridization sequence in adapters. As used herein, whole-genome amplification refers to amplifying with the goal of amplifying the entire genome. In some embodiments, a genome is amplified relative to the original amount of the genome present in a sample by at least 2-fold (e.g., at least 2-fold, at least 5-fold, at least 10-fold, at least 50-fold, at least 100-fold, at least 200-fold, at least 500-fold, at least 1000-fold, at least 2000-fold, or at least 5000-fold). In some embodiments, whole-genome amplification is performed using a standard displacement whole genome amplification, for example, by using Phi29 polymerase. Performing whole-genome amplification at this step in the method enables generating higher amounts of DNA that can be used repeatedly for additional applications.

### Sequencing and Post-Sequencing Analysis

In some embodiments, the methods described herein comprise sequencing the amplified regions between repeat elements or repeat element/s and a common hybridization sequence in an adapter. Non-limiting examples of sequencing methods include Sanger sequencing, Next-Generation sequencing, and massively parallel sequencing (MPS).

In some embodiments, post-sequencing analysis is performed to identify amplification and sequencing errors. In some embodiments, single-stranded or double-stranded consensus techniques are performed using a UMI to identify amplification and sequencing errors in sequencing data obtained from the amplified regions obtained using any one of the methods disclosed herein. Smith et al. (Genome Res., 27, 491-499) describes such methods, and is incorporated herein by reference in its entirety. In some embodiments, amplification and sequencing errors are identified using a UMI that is formed at the junction of fragments of genomic DNA that are formed as a result of random ligation. In some embodiments, a UMI at the junction of two fragments in a concatemer comprises at least two base pairs (e.g., at least 2, at least 3, at least 4, etc.) of each fragment on either side of a junction. For example, if a first and second fragment are ligated together randomly so that the 3′ end of the first fragment and the 5′ end of the second fragment form a junction, and the 3′ end of the first fragment comprises the sequence AGGCT and the 5′ end of the second sequence comprises TTATC, then a UMI may be defined as CTTT. It should be noted that a UMI may comprise a different number of base pairs for the fragments that make up a junction. For example, a UMI for the above-described first and second fragments may be GCTTT.

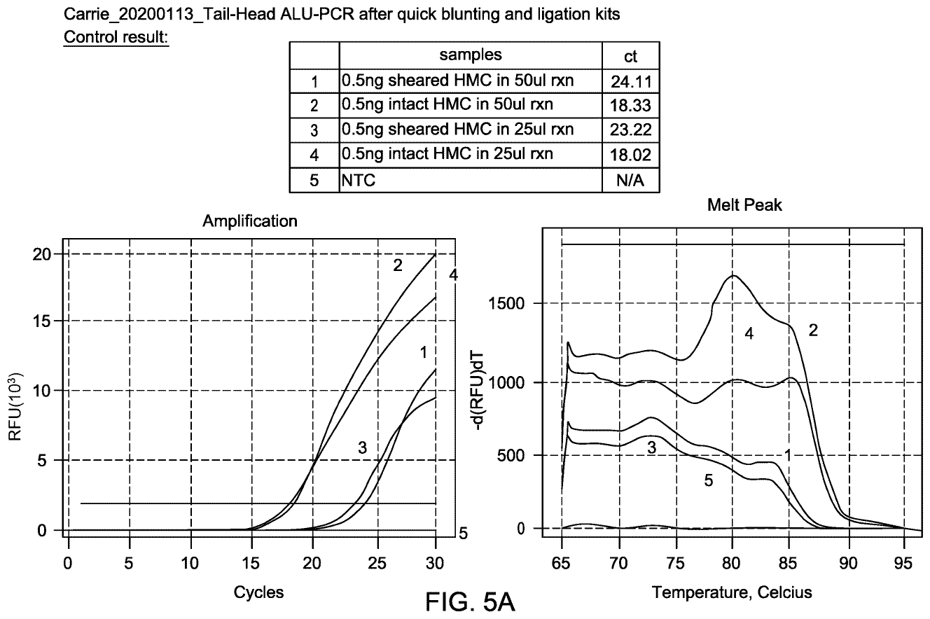

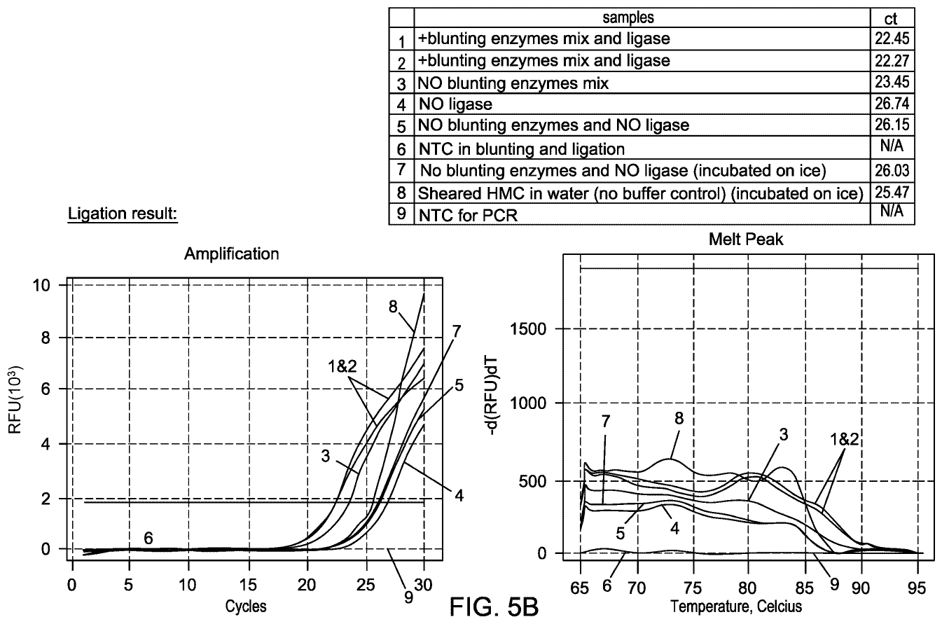

In some embodiments, subjecting the nucleic acid fragments in a sample to random ligation conditions prior to DNA amplification results in an increase in amplifiable nucleic acid by at least 2, at least 3, at least 4, or at least 5 PCR cycles compared to a method comprising performing DNA amplification without subjecting the nucleic acid fragments to random ligation conditions (see, e.g., FIGS. 5A-5B).

### A Method of Enriching Regions of a Genome Using Primers Complementary to Repeat Elements and Common Hybridization Sequences on Adapters

Provided herein are methods of enriching regions of a genome using, instead of two or more repeat elements, one or more repeat elements and a common hybridization sequence on an adapter. In some embodiments, a method uses (1) a sample containing double-stranded fragments of genomic nucleic acid; and (2) double-stranded adapters, wherein one end of the adapter comprises a first 3′ dT overhang and is 5′ phosphorylated and the other end of the adapter is blunted or comprises a second 3′ dT overhang and is 5′ phosphorylated, and wherein each adapter comprises a common hybridization sequence and a unique molecular identifier (UMI) that is between the first or second dT overhang and the common hybridization sequence and is different from the UMI on any other adapter in the sample.

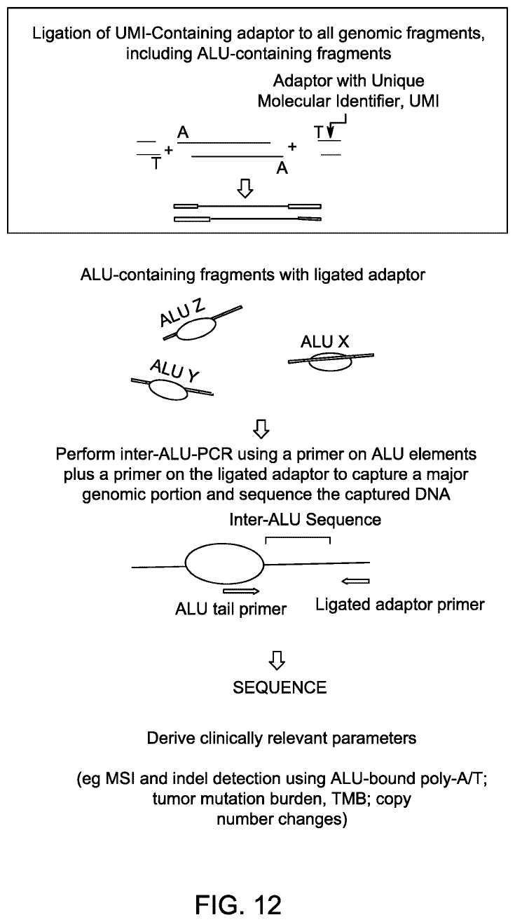

In some embodiments, a method using a common hybridization sequence comprises creating blunt ends on the fragments of genomic nucleic acid; adding a single dA at the 3′ ends of the blunted fragments to create fragments with a 3′ dA overhang on both strands of the fragments; adding to the sample double-stranded adapters with a common hybridization sequence and a UMI; applying ligation conditions to the sample to form a plurality of double-stranded adapter ligated fragments, each adapter ligated fragment comprising a first strand and a second strand and an adapter on each end of the fragment, wherein at least some of the adapter ligated fragments comprise a repeat element; and performing amplification using a pair of primers, wherein a first primer of the pair of primers is complementary to a sequence in the first strand within the repeat element and the second primer of the pair of primers is complementary to a sequence in the second strand within the common hybridization sequence on the adapters, wherein performing the amplification amplifies the nucleic acid between the far ends of the repeat element and the common hybridization sequence on the adapters. See for example, FIG. 12.

In some embodiments, a method using one or more repeat elements and a common hybridization sequence results in regions of the genome being amplified by at least 100-fold, at least 200-fold, at least 500-fold, at least 1000-fold, at least 2000-fold, or at least 5000-fold relative to the original amount (before ligation and amplification) of genome present in the sample.

In some embodiments, at least some of the adapter ligated fragments have a repeat element within their sequence.

In some embodiments, the adapters have a 3′ deoxythymidine (dT) on one end of each of the adapters and are 5′ phosphorylated. In some embodiments, one end of each of the adapters is blunt. In some embodiments, the adapters have a 3′ dT overhang on both ends of each of the adapters and both ends of the adapters are 5′ phosphorylated. In some embodiments, the adapters are 4-20 bp long (e.g., 4-20 bp, 5-18 bp, 6-16 bp, 7-14 bp, 8-12 bp, or 9-10 bp long). In some embodiments, each adapter has a unique molecular identifier (UMI) that is different from that on any other adapter in the sample. In some embodiments, the UMI is at least 2 bp long. In some embodiments, the UMI is at least 3 bp, at least 4 bp, at least 5 bp, or at least 10 bp long. In some embodiments, the adapters comprise a 1 or 2 bp mismatch in the center of the adapter. In some embodiments, the DNA fragments are 5′ phosphorylated.

In some embodiments, the adapter ligated fragments have a first strand and a second strand. In some embodiments, a plurality of nucleic acid fragments in a sample have an adapter ligated to each end of the fragments. In some embodiments, a plurality of nucleic acid fragments in a sample have an adapter ligated to only one end. In some embodiments, at least some of the adapter-ligated fragments comprise at least one repeat element.

In some embodiments of any one of the methods described herein, regions of genomic nucleic acid are amplified relative to unamplified regions of the genome by at least 2-fold, at least 5-fold, at least 10-fold, at least 50-fold, at least 100-fold, at least 200-fold, at least 500-fold, at least 1000-fold, at least 2000-fold, or at least 5000-fold. In some embodiments, amplification includes performing touchdown PCR. Touchdown PCR is a PCR method in which higher annealing temperatures are used in the earlier cycles of PCR to anneal the primers to the nucleic acid template, and the annealing temperature is progressively decreased in subsequent PCR cycles. Touchdown PCR avoids the amplification of nonspecific sequences. In some embodiments, 2-20 cycles of touchdown PCR (e.g., 2-20, 3-18, 4-16, 5-14, 6-12, or 7-10 cycles) are performed prior to performing COLD-PCR and/or step-up PCR to preferentially amplify repeat elements with a deletion.

In some embodiments, DNA amplification is performed by isothermal DNA amplification. Some examples of isothermal DNA amplification are transcription mediated amplification, nucleic acid sequence-based amplification, strand displacement amplification, rolling circle amplification, loop-mediated isothermal amplification, isothermal multiple displacement amplification, helicase-dependent amplification, single primer isothermal amplification, recombinase polymerase amplification, and circular helicase-dependent amplification (Gill et al., Nucleosides Nucleotides Nucleic Acids, 27, 224-243).

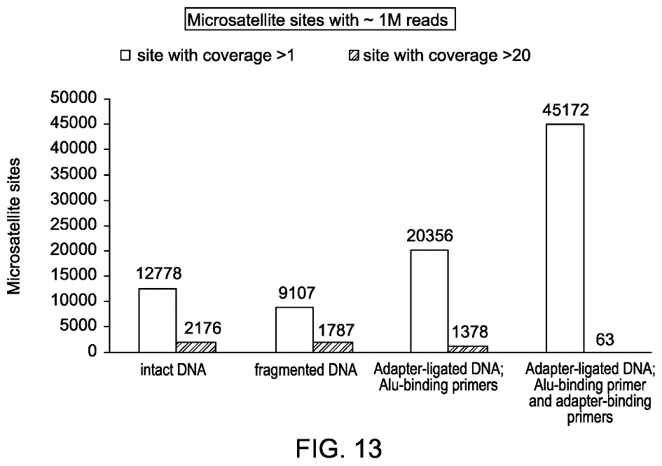

In some embodiments, the concentration of the primer complementary to the repeat element is at least 2 times (e.g., at least 3 times, at least 4 times, at least 5 times, or at least 10 times) higher than the concentration of the primer complementary to the common hybridization sequence on the adapter. In some embodiments, performing amplification using primers that are complementary to a repeat element and a common hybridization sequence on an adapter results in an increase in the number of repeat elements captured by amplification that is at least 2-fold (e.g., at least 5-fold, at least 6-fold, at least 7-fold, at least 8-fold, at least 9-fold, at least 10-fold, at least 11-fold, at least 12-fold, at least 13-fold, at least 14-fold, or at least 15-fold) compared to performing amplification using primers that are complementary to two or more repeat elements. See, e.g., FIG. 13.

In some embodiments, the method further comprises sequencing the amplified regions between as described above. In some embodiments, post-sequencing analysis is performed to identify amplification and sequencing errors as described above.

### Applications and Biological Endpoints

In some embodiments, the methods described herein may be used in determining the number of mutations in the amplified regions of genomic nucleic acid. Analyzing the number of mutations provides information related to mismatch repair deficiency or total mutation burden. Mismatch repair deficiency results when mismatch repair pathways in a cell are less efficiently capable of correcting DNA replication and genetic recombination errors. Mismatch repair deficiency may provide an indication of the presence of a disease (e.g., cancer). Total mutation burden describes the total number of mutations in the DNA of cancer cells. Determining mutation burden may be useful in determining the optimal course of treatment for a specific cancer.

In some embodiments, the methods described herein may be used in determining the number of insertions or deletions in homopolymers or heteropolymers in the amplified regions of genomic nucleic acid. Analyzing the number of insertions or deletions provides an indication of microsatellite instability. Homopolymers are a type of simple sequence repeat in which a single nucleotide is repeated (e.g., poly(dA), poly(dT), poly(dG), or poly(dC). Homopolymers may be greater than 3 nucleotides in length (e.g., greater than 3 nucleotides, greater than 10 nucleotides, greater than 50 nucleotides, or greater than 100 nucleotides in length). Heteropolymers are repeating sequences made up of more than one single nucleotide (e.g., two different nucleotides, three different nucleotides, or four or more different nucleotides).

In some embodiments, the methods described herein may be used in determining the number of copies of a gene of interest in the amplified regions of genomic nucleic acid. Analyzing the number of copies of a gene of interest provides an indication of the presence of a disease (e.g., cancer).

In some embodiments, the methods described herein may be used for determining the number of methylated forms of a gene of interest (e.g., DNA methylation) in the amplified regions of genomic nucleic acid. DNA methylation is a process by which methyl groups are added to a DNA molecule. DNA methylation may repress gene expression, or it may change the activity of a nucleic acid sequence. The methylation state of a nucleic acid sequence can provide information about the presence of a disease (e.g., cancer).

In some embodiments, the methods described herein may be used for determining the number of a short tandem repeat in the amplified regions of genomic nucleic acid. The number of a short tandem repeat may be compared to the number of the short tandem repeat in a reference sample. The number of a short tandem repeat can provide information about the presence of a disease (e.g., cancer).

### EXAMPLES

### Example 1: Random Ligation Followed by Inter-Alu-PCR

Described here are examples using random ligation of fragments of genomic DNA and inter-Alu-PCR as a means of amplifying nucleic acid between Alu elements. It should be understood that Alu elements are merely an example of a repeat element that can be used in the methods and that it can be replaced with any other repeat element, such as those described in this disclosure.

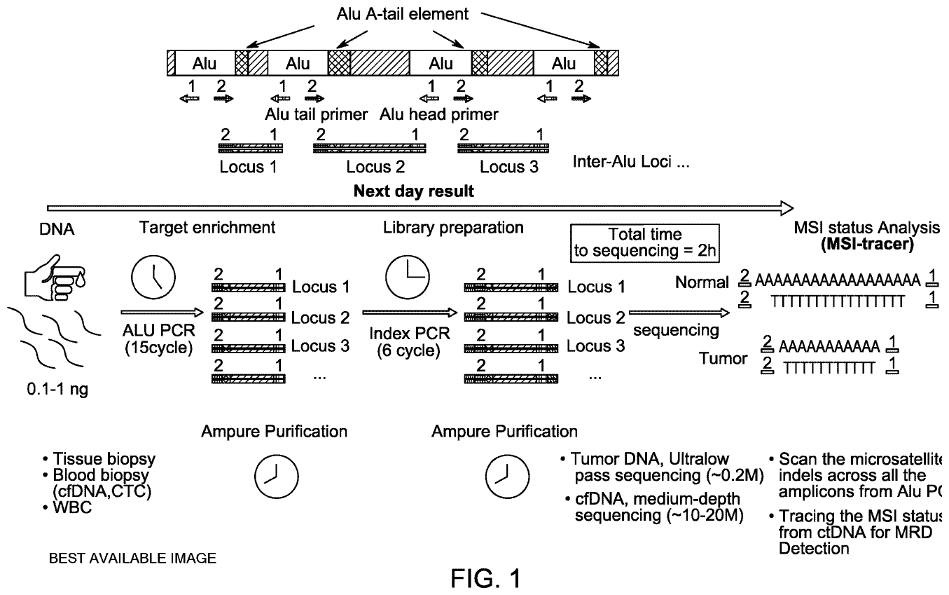

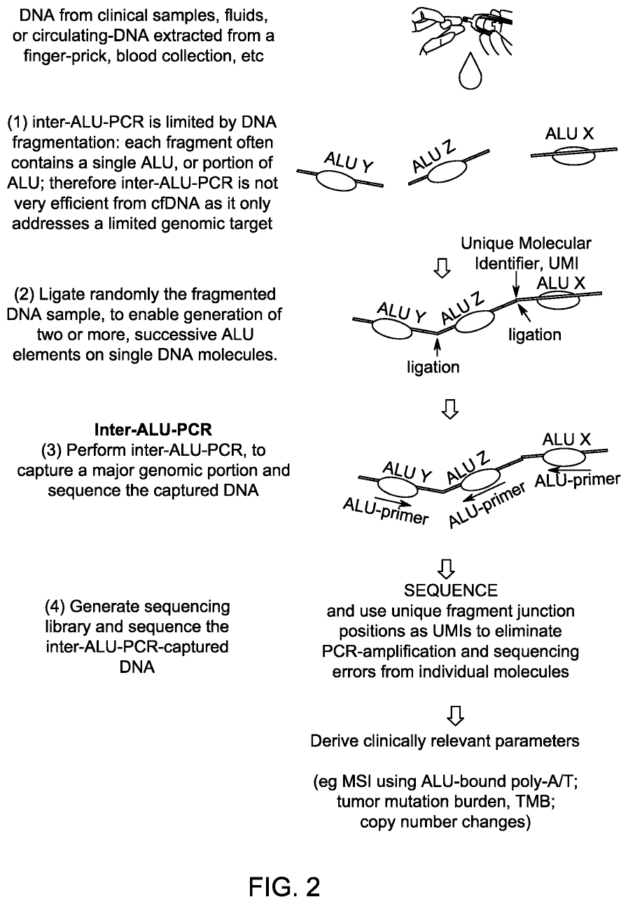

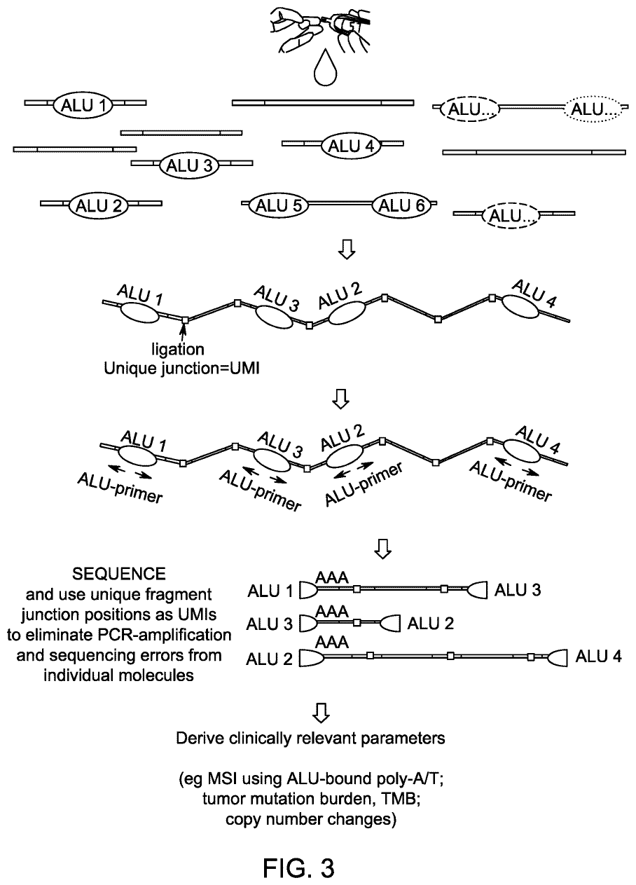

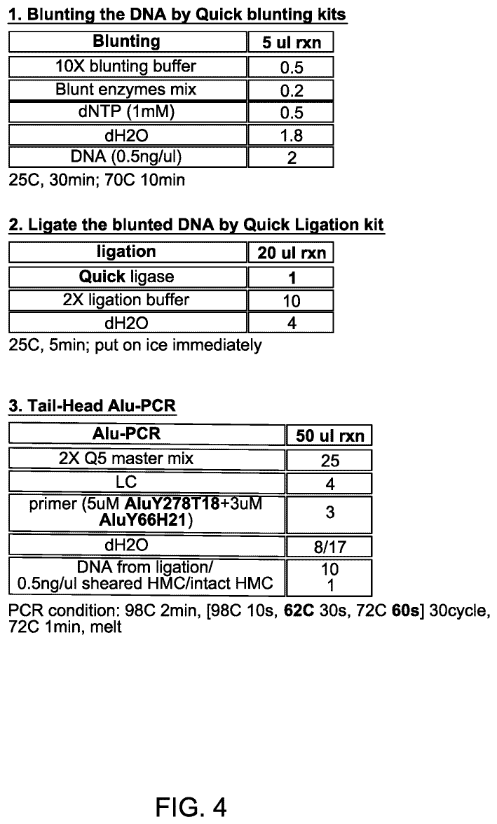

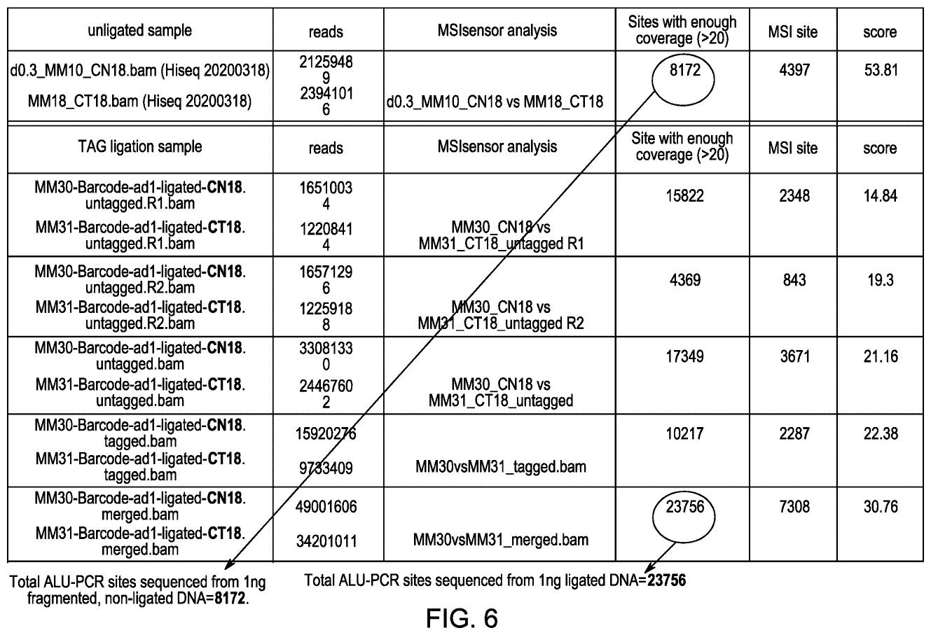

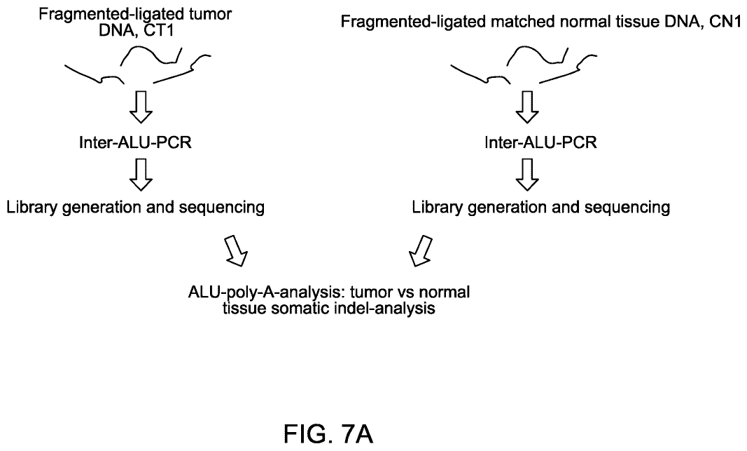

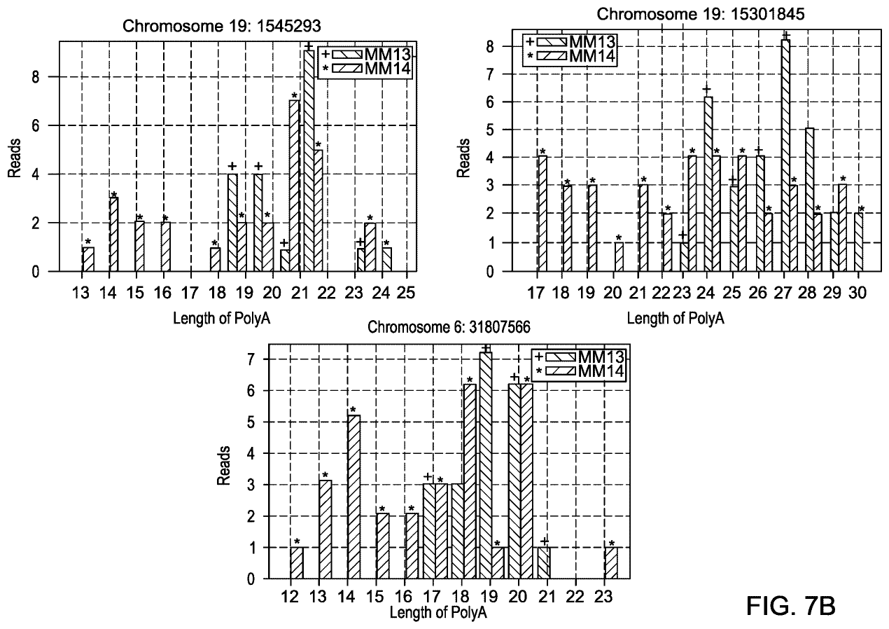

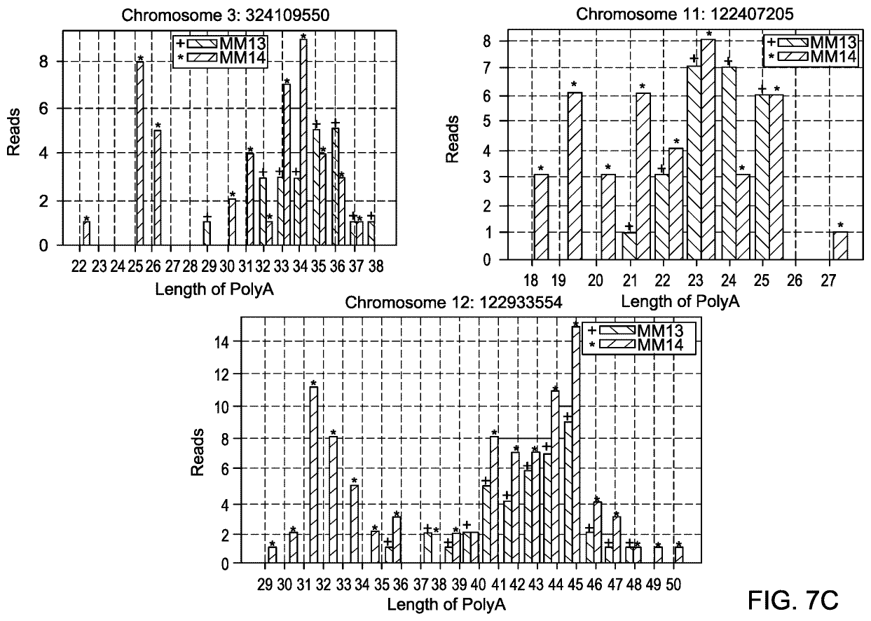

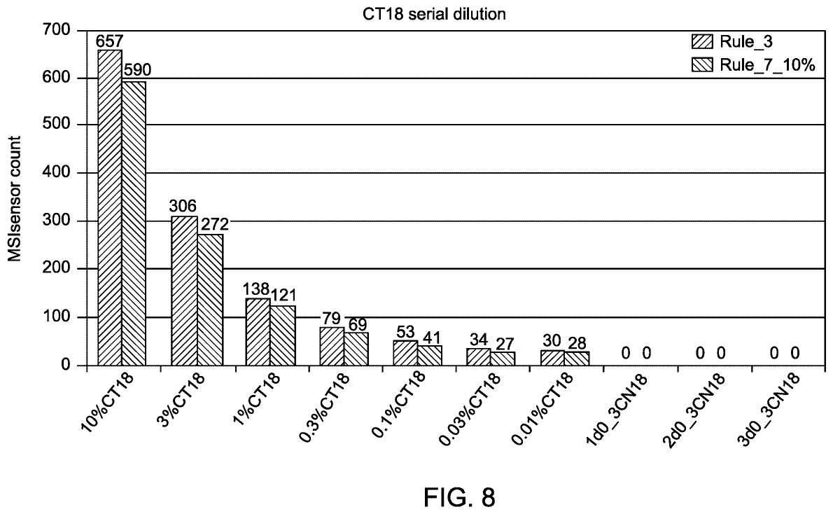

FIG. 2 and FIG. 3 show random-ligation-based inter-Alu-PCR, an approach to capture a bigger portion of the Alu elements in the genome than those captured by inter-Alu-PCR from intact DNA, or by inter-Alu-PCR from non-ligated cfDNA. To this end, if the DNA obtained is intact (e.g., DNA from biopsies), one may apply random fragmentation (e.g., by enzymatic Shearase techniques), then re-ligate DNA randomly as illustrated in FIG. 1 and apply inter-Alu-PCR to capture a bigger genomic portion as compared to direct inter-Alu-PCR from intact genomic DNA. FIG. 3 illustrates a method wherein some fragments comprise no repeat elements (at least the repeat element that is used for amplification). FIG. 4 provides an example of reagents and conditions that can be used to perform a method that was used to generate the data in FIGS. 5A-5B. FIGS. 5A-5B demonstrate that following inter-Alu-PCR, the amount of amplified DNA increases because of generating longer DNA fragments that contain more than one Alu element in proximity. Specifically, the real time PCR threshold when applying Alu-PCR is similar or lower to the one obtained with intact DNA, indicating that ligation has indeed formed longer fragments that can be amplified efficiently in Alu-PCR reactions. Following random ligation, the amount of amplifiable DNA increases by 4 PCR cycles, i.e., about 16-fold, as opposed to non-ligated fragmented DNA. Therefore, the amount and diversity of inter-Alu sequences captured increases. There was no difference between the 25 μl and 50 μl reactions. The cycle threshold (ct) difference between sheared HMC and intact HMC is still in the range of 5-6 cycles. However, both sheared HMC and intact HMC showed late amplification compared to previous experiments (DNA may be degraded during freeze thaw). Similar ct (˜26 cycles) were observed in S4 (no ligase control), S5 (no blunting enzyme and no ligase), and S7 (no blunting enzymes and no ligase (incubated on ice)). Compared to S4, S5, and S7, S1 (+enzyme blunting mix and ligase) and S2 (+enzyme blunting mix and ligase) gave a ct of about 3-4 cycles earlier. Also, compared to standard 0.5 ng sheared HMC (24.11 cycles), S1 and S2 gave act of about 1.6 cycles earlier, indicating that the blunting and ligation work. Compared to S4, S5, and S7, S3 (no blunting enzyme mix) showed a ct of about 3 cycles earlier, indicating that some of the sheared HMC may be blunted. S8 (sheared HMC in water (no buffer control) incubation on ice) during the process of blunting and ligation has a 1.36 cycle delay compared to standard sheared HMC (24.11 cycles). However, this degradation is not from the blunting and ligation buffer since the cycles of S8 is almost the same compared to S7. Results from S7 and S8 also indicate that the blunting and ligation buffer does not affect the Alu-PCR. FIG. 6 indicates the results of sequencing Alu-PCR products from fragmented DNA versus ligated DNA. The number of Alu-PCR targets detected following inter-ligation of DNA is several times higher compared to fragmented DNA. FIGS. 7A-7C depicts examples of side-by-side comparisons of polyAs sequenced in tumor versus corresponding normal tissue using fragmented-ligated DNA as per FIG. 2. The indels in the tumor are evident and enable detection of the presence of the tumor even at extremely low fractions of tumor-originating DNA. For example, in FIG. 8, ratios as low as 0.01% tumor DNA to normal DNA can readily be distinguished from pure normal tissue DNA.

If instead of Alu-elements, one wishes to amplify different repeat elements (e.g., long interspersed elements 1 (Line1 or L1 elements) via PCR (Belic et al., Clin Chem, 61, 838-849; Kopera et al., Methods Mol Biol, 1400, 339-355; Kinde et al., PLoS One, 7, e41162); or inter-simple sequence repeat (ISSR) PCR (Suyama et al., Sci Rep, 5, 16963); or any other long or short sequence repeat in the human, mammalian or plant genome) the same approach can be applied: fragment the DNA (if not already biologically fragmented, like cfDNA), randomly re-ligate DNA, and then apply inter-repeat-sequence PCR using primers hybridizing to the repeat elements, or to combinations of repeat elements (e.g., Alu primer combined with L1 primer, etc.).

Random ligation between DNA fragments can be applied in more than one way. If the DNA is double stranded, such as the majority of DNA circulating in blood, then a standard blunting of DNA ends using T4 DNA polymerase, followed by blunt-end ligation, and then followed by inter-Alu-PCR can be applied, as shown in FIG. 2.

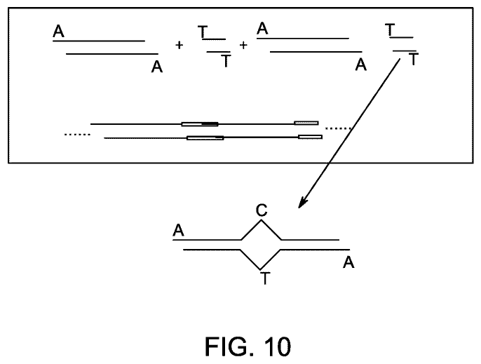

FIG. 9 shows a ligation approach that includes creating blunted DNA fragments, then adding a single dA at the 3′ end of each fragment via dA-tailing, and then introducing short double stranded DNA adapters (also referred to herein as tags) that are 5′ phosphorylated on both sides and contain a protruding dT on both 3′ ends. Using a standard ligation reaction in the presence of these tags or adapters, extensive cross-ligation of the fragmented DNA targets is enabled, and it is ensured that each DNA fragment is ligated on both sides with tags/adapters prior to becoming ligated with another DNA fragment. In this way, the long concatemers formed have a well-defined structure, and each fragment is encompassed by two adapters. Additionally, when dA tailing and ligation to dT adapters is used, self-ligation (circularization) of single DNA fragments is prevented since the two ends of each fragment are non-complementary following dA tailing. The tags comprise complementary oligonucleotides of 4-20 bp size with a protruding dT at the 3′ ends and phosphorylated 5′ ends. FIG. 10 shows that these adapters may comprise a 1-2 nucleotide mismatch (e.g., at the center). This enables distinguishing the top DNA strand from the bottom DNA strand during subsequent sequencing and can be used for duplex-sequencing corrections in bioinformatic analysis.

Finally, another approach for ligation is to denature the DNA and then apply single strand DNA ligase to create concatemers of single stranded DNA, following which inter-Alu-PCR can be applied. To prevent formation of DNA circular molecules during ligation, as opposed to the desired DNA concatemers, 7-15% of polyethylene glycol (PEG) can be included in the solution during the ligation step. PEG reduces the ‘flexibility’ of single stranded DNA, thereby reducing self-circularization.

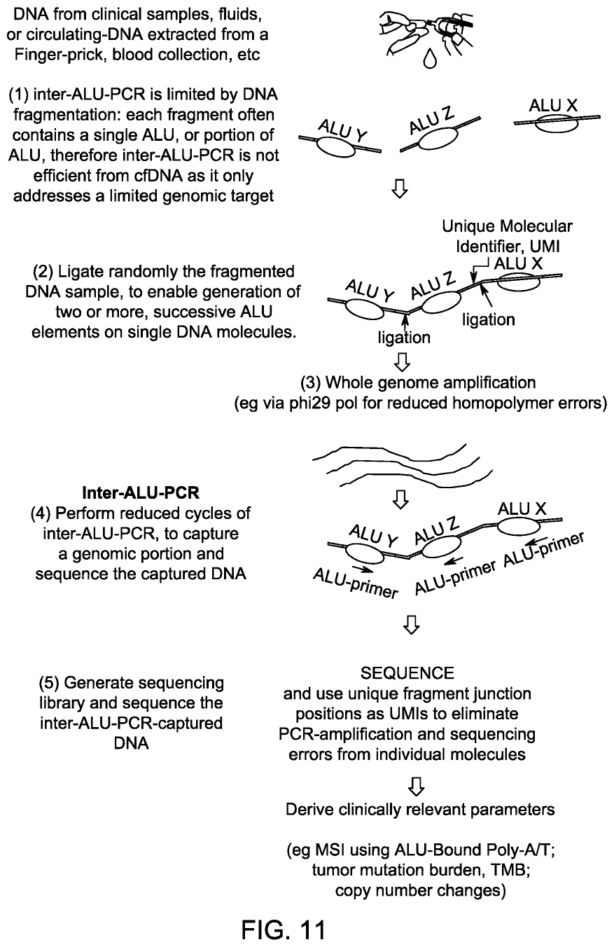

FIG. 11 shows that one optional additional step following ligation of fragmented DNA is to apply whole genome amplification (e.g., via strand displacement) prior to using the DNA for Alu-PCR. This step enables generating higher amounts of DNA that can be used repeatedly for additional applications beyond the present Alu-PCR. To this end, a standard displacement whole genome amplification using Phi29 polymerase can be applied to the ligation-formed concatemers following ligation. This can then be followed by the same Alu-PCR approach as shown in FIG. 2.

### Example 2: Adapter-Ligation-Mediated Alu-PCR

FIG. 12 shows another alternative for performing the ligation step and inter-Alu-PCR (or amplification using any other method and any other repeat element/s in the genome) in a manner that captures higher numbers of Alu elements. In the approach illustrated in FIG. 12, a standard ligation step using UMI-containing adapters is first performed. One method of ligation is to blunt end the fragments, followed by adding a dA overhang on the 3′ ends of the fragments, and then ligating with adapters having a 3′ dT overhang on one or both ends. The adapter comprises a common hybridization sequence that can be used for amplification and may also comprise a UMI. The ligation step is then followed by inter-Alu-PCR using a forward primer anchored on Alu (preferably an Alu-tail primer) plus a reverse primer anchored on the common hybridization sequence on the ligated adapter. This approach captures a major portion of the Alu-elements in the genome, thereby providing a bigger target for information-rich sequencing. The Alu-PCR conditions using the Alu-tail primer plus ligated adapter are regulated to provide high specificity for Alu elements. In a preferred set of conditions, the concentration of Alu-tail primer is set to be 5× the concentration of adapter-primer. In addition, during PCR a 10-cycle touchdown PCR program is applied, followed by 25 cycles of regular PCR to enable higher specificity for Alu elements. Following generation of a sequencing library and paired end sequencing, standard alignment is applied to match sequences to the standard human genome. Additionally, during bioinformatic analysis the UMI incorporated in the ligated adapter is used to eliminate PCR errors and other noise-producing artifacts.

Alternatively, the DNA amplification protocol in FIG. 12 may follow a format that enables selective enrichment of Alu elements with poly-adenine tails containing large deletions. For example, the amplification protocol may follow COLD-PCR conditions (Li et al., Nat Med, 14, 579-584), upon which selected denaturation temperature conditions lead to preferential amplification of deletion-containing DNA and elimination of the wild-type form of DNA.

Alternatively, instead of applying PCR-based amplification conditions, an isothermal amplification of tag-ligated Alu sites can be applied (Gill et al., Nucleosides Nucleotides Nucleic Acids, 27, 224-243). Isothermal amplification can take place by using the same primers described above, under conditions enabling amplification, transcription mediated amplification, nucleic acid sequence-based amplification, strand displacement amplification, rolling circle amplification, loop-mediated isothermal amplification of DNA, isothermal multiple displacement amplification, helicase-dependent amplification, single primer isothermal amplification, recombinase polymerase amplification, or circular helicase-dependent amplification (Gill et al., Nucleosides Nucleotides Nucleic Acids, 27, 224-243). An advantage of using isothermal methods of amplification is that in many cases the polymerase-introduced errors during amplification of poly-adenine homopolymers (known as ‘stutter’) is reduced relative to conventional PCR (Daunay et al., Nucleic Acids Res, 47, e141).

FIG. 13 shows experimental data comparing the number of different Alu elements that can be amplified by the various approaches: direct inter-Alu-PCR using an Alu-tail primer plus an Alu-head primer, applied to either intact DNA or fragmented DNA/cfDNA; adapter-ligated DNA followed by Alu-PCR using two Alu-binding primers as shown in FIG. 9; and finally, adapter-ligated DNA followed by inter-Alu-PCR using one Alu-binding primer and one adapter-binding primer as shown in FIG. 12. The approaches described in the present disclosure lead to capture and sequencing of many more Alu-elements than the direct Alu-PCR approach. In turn, this translates to superior, information-rich sequencing for clinical applications like MSI-identification, tumor mutational burden detection, copy number changes, etc.

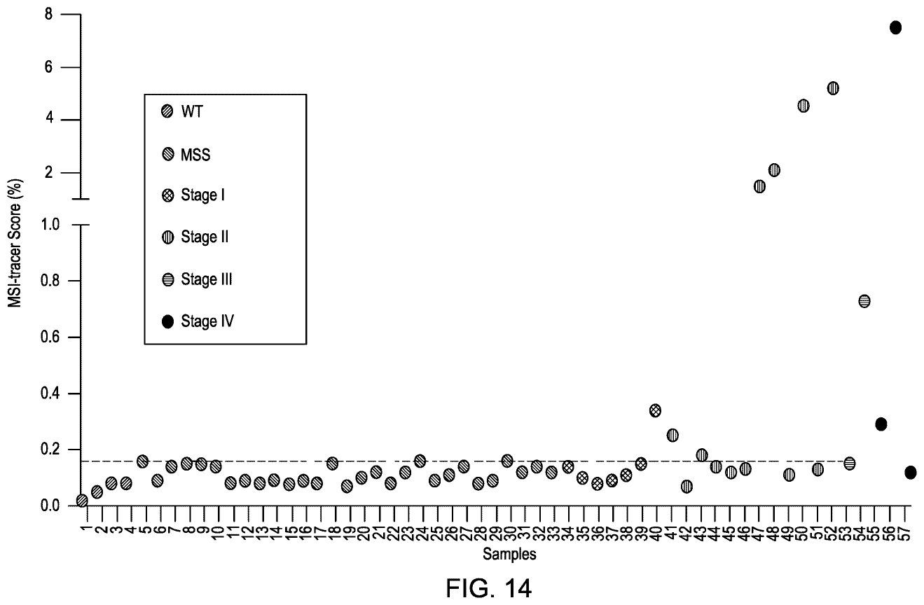

An example applying the approach shown in FIGS. 12 to 1 ng of circulating DNA obtained from colon cancer patients with known MSI-status of their tumors, versus cancer stage, is shown in FIG. 14. By using just 1 ng of circulating DNA, it is possible to diagnose whether the tumor is MSI-positive in 40-60% of cases for stages II-IV, with 100% specificity. Stage I tumors have a lower (20%) detection sensitivity. It is anticipated that increasing the amount of input DNA to 10 ng will increase the sensitivity of detection for MSI-positive tumors.

### Example 3: Biological Endpoints and Applications

Multiple biological endpoints of clinical significance can be derived following any one of the methods described herein, e.g., copy number analysis (L1), methylation analysis (L1), inherited or somatic mutation analysis (Alu) and others (Mei et al, BMC Genom, 12, 564).

Genome-wide homopolymer indel detection: One significant advantage with Alu-element amplification, as opposed to other repeat elements that do not have a polyA tail is that Alu elements contain poly-adenine tails (polyA tails) towards the end of the sequence. Long poly-As are homopolymers that often undergo insertions or deletions (indels) of several A's, especially under conditions of mismatch repair causing micro-satellite instability (MSI-high or simply MSI). MSI is an indicator of response to immunotherapy, with MSI-high patients showing better response. As shown in FIGS. 5A-5B, detection of microsatellite instability by focusing on indels occurring at the poly-A tails of Alu can be demonstrated by applying inter-Alu-PCR. Therefore, the present disclosure for enhanced amplification of genomic fractions following the approach shown in FIG. 2, FIG. 9, or FIG. 12, enables improved analysis of MSI or other endpoints from intact or fragmented DNA using minute amounts of starting DNA (FIG. 3 shows use of 1 ng, but much less down to 10 pg can also be used, as shown in the data).

In addition to detection of micro-satellite instability in tumor samples undergoing mismatch repair deficiency, which comprise a small percentage of all tumors, the present method is so sensitive that it may also detect indels in non-repair deficient samples (‘microsatellite stable’, or ‘MSS’ samples). MSS samples generate indels at lower frequency than MSI-high samples, and their indels are smaller as compared to MSI-high samples, but they can still be detected using the methods described in the present disclosure.

Another potential application of Alu in any one of the methods described herein is measuring Tumor Mutational Burden (TMB). TMB is defined as the number of somatic mutations per mega-base of DNA, as identified via sequencing, and is an important biomarker for (positive) patient response to immunotherapy. To perform accurate detection of TMB, even at very low allelic frequency of mutations (e.g., 0.1% or less, such as mutations in cfDNA), an error correction method is required. In embodiments shown in FIG. 2, FIG. 9, and FIG. 12, the error correction is provided by the random junction formed upon random ligation between DNA fragments, or by the ligated UMI-containing adapter.

Another application of any one of the methods disclosed herein is in forensics, where short tandem repeat (STR) analysis is used from fragmented DNA in crime scenes. Forensic STR analysis is limited by the quality and quantity of DNA. Accordingly, the current protocols that improve repeat element amplification can improve analysis.

Regarding applications in liquid biopsy, the enhanced detection of inter-Alu-elements following random inter-ligation illustrated in FIG. 2, FIG. 9, or FIG. 12 enables cfDNA analysis from minute amounts of blood, such as those obtained from a finger-prick. Accordingly, cfDNA analysis can be performed using minimally invasive procedures that can conceivably be done by untrained individuals at home as opposed to the doctor's office. Further, these can be performed more frequently than standard blood draws that use 10 ml blood or more, thereby enabling better monitoring of tumor status at regular time-points, and potentially improving detection of minimal residual disease. Finally, the ability to perform cfDNA analysis from finger-pricks may also enable early cancer detection in certain classes of high-risk individuals, e.g., Lynch syndrome patients, etc.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

### REFERENCES

[1] Diehl F, Schmidt K, Choti M A, Romans K, Goodman S, Li M, Thornton K, Agrawal N, Sokoll L, Szabo S A, Kinzler K W, Vogelstein B, Diaz L A, Jr.: Circulating mutant DNA to assess tumor dynamics. Nat Med 2008, 14:985-90.
[2] Thierry A R, Mouliere F, El Messaoudi S, Mollevi C, Lopez-Crapez E, Rolet F, Gillet B, Gongora C, Dechelotte P, Robert B, Del Rio M, Lamy P J, Bibeau F, Nouaille M, Loriot V, Jarrousse A S, Molina F, Mathonnet M, Pezet D, Ychou M: Clinical validation of the detection of KRAS and BRAF mutations from circulating tumor DNA. Nat Med 2014, 20:430-5.
[3] Newman A M, Bratman S V, To J, Wynne J F, Eclov N C, Modlin L A, Liu CL, Neal J W, Wakelee H A, Merritt R E, Shrager J B, Loo B W, Jr., Alizadeh A A, Diehn M: An ultrasensitive method for quantitating circulating tumor DNA with broad patient coverage. Nat Med 2014, 20:548-54.
[4] Bettegowda C, Sausen M, Leary R J, Kinde I, Wang Y, Agrawal N, Bartlett B R, Wang H, Luber B, Alani R M, Antonarakis E S, Azad N S, Bardelli A, Brem H, Cameron J L, Lee C C, Fecher L A, Gallia G L, Gibbs P, Le D, Giuntoli R L, Goggins M, Hogarty M D, Holdhoff M, Hong S M, Jiao Y, Juhl H H, Kim J J, Siravegna G, Laheru D A, Lauricella C, Lim M, Lipson E J, Marie S K, Netto G J, Oliner K S, Olivi A, Olsson L, Riggins G J, Sartore-Bianchi A, Schmidt K, Shih l M, Oba-Shinjo S M, Siena S, Theodorescu D, Tie J, Harkins T T, Veronese S, Wang T L, Weingart J D, Wolfgang C L, Wood L D, Xing D, Hruban R H, Wu J, Allen P J, Schmidt C M, Choti M A, Velculescu V E, Kinzler K W, Vogelstein B, Papadopoulos N, Diaz LA, Jr.: Detection of circulating tumor DNA in early- and late-stage human malignancies. Sci Transl Med 2014, 6:224ra24.
[5] Diehl F, Li M, Dressman D, He Y, Shen D, Szabo S, Diaz L A, Jr., Goodman S N, David K A, Juhl H, Kinzler K W, Vogelstein B: Detection and quantification of mutations in the plasma of patients with colorectal tumors. Proc Natl Acad Sci U S A 2005, 102:16368-73.
[6] Schwaederle M, Husain H, Fanta P T, Piccioni D E, Kesari S, Schwab R B, Patel S P, Harismendy O, Ikeda M, Parker B A, Kurzrock R: Use of Liquid Biopsies in Clinical Oncology: Pilot Experience in 168 Patients. Clin Cancer Res 2016, 22:5497-505.
[7] Roschewski M, Dunleavy K, Pittaluga S, Moorhead M, Pepin F, Kong K, Shovlin M, Jaffe E S, Staudt L M, Lai C, Steinberg S M, Chen C C, Zheng J, Willis T D, Faham M, Wilson W H: Circulating tumour DNA and CT monitoring in patients with untreated diffuse large B-cell lymphoma: a correlative biomarker study. Lancet Oncol 2015, 16:541-9.
[8] Mei L, Ding X, Tsang S Y, Pun F W, Ng S K, Yang J, Zhao C, Li D, Wan W, Yu C H, Tan T C, Poon W S, Leung G K, Ng H K, Zhang L, Xue H: AluScan: a method for genome-wide scanning of sequence and structure variations in the human genome. BMC Genomics 2011, 12:564.
[9] Konkel M K, Batzer M A: A mobile threat to genome stability: The impact of non-LTR retrotransposons upon the human genome. Semin Cancer Biol 2010, 20:211-21.
[10] Belic J, Koch M, Ulz P, Auer M, Gerhalter T, Mohan S, Fischereder K, Petru E, Bauernhofer T, Geigl J B, Speicher M R, Heitzer E: Rapid Identification of Plasma DNA Samples with Increased ctDNA Levels by a Modified FAST-SeqS Approach. Clin Chem 2015, 61:838-49.
[11] Kopera H C, Flasch D A, Nakamura M, Miyoshi T, Doucet A J, Moran J V: LEAP: L1 Element Amplification Protocol. Methods Mol Biol 2016, 1400:339-55.
[12] Kinde I, Papadopoulos N, Kinzler K W, Vogelstein B: FAST-SeqS: a simple and efficient method for the detection of aneuploidy by massively parallel sequencing. PLoS One 2012, 7:e41162.
[13] Suyama Y, Matsuki Y: MIG-seq: an effective PCR-based method for genome-wide single-nucleotide polymorphism genotyping using the next-generation sequencing platform. Sci Rep 2015, 5:16963.
[14] Li J, Wang L, Mamon H, Kulke M H, Berbeco R, Makrigiorgos G M: Replacing PCR with COLD-PCR enriches variant DNA sequences and redefines the sensitivity of genetic testing. Nat Med 2008, 14:579-84.
[15] Gill P, Ghaemi A: Nucleic acid isothermal amplification technologies: a review. Nucleosides Nucleotides Nucleic Acids 2008, 27:224-43.
[16] Daunay A, Duval A, Baudrin L G, Buhard O, Renault V, Deleuze J F, How-Kit A: Low temperature isothermal amplification of microsatellites drastically reduces stutter artifact formation and improves microsatellite instability detection in cancer. Nucleic Acids Res 2019, 47:e141.
[17] Roy-Engel A M, Salem A-H, Oyeniran O O, Deininger L, Hedges D J, Kilroy G E, Batzer M A, Deninger P L: Active Alu Element “A-Tails”: Size Does Matter. Genome Res 2002, 12:1333-1344.
[18] Viswanathan M, Sangiliyandi G, Vinod S S, Mohanprasad B K C, Shanmugam G: Genomic Instability and Tumor-specific Alterations in Oral Squamous Cell Carcinomas Assessed by Inter-(Simple Sequence Repeat) PCR. Clin Cancer Res 2003, 9:1057-1062.
[19] Hosono S, Faruqi A F, Dean F B, Du Y, Sun Z, Wu X, Du J, Kingsmore S F, Egholm M, Lasken R S: Unbiased Whole-Genome Amplification Directly From Clinical Samples. Genome Res 2003, 13:954-964.
[20] Smith T, Heger A, Sudbery I: UMI-tools: modeling sequencing errors in Unique Molecular Identifiers to improve quantification accuracy. Genome Res 2017, 27:491-499.

What is claimed is:

1. A method of enriching portions of a genome in a sample of genomic nucleic acid, the method comprising:
(a) providing a sample containing double-stranded fragments of genomic nucleic acid;
(b) adding to the sample double-stranded adapters, wherein each adapter comprises a common hybridization sequence;
(c) applying ligation conditions to the sample to form a plurality of double-stranded adapter ligated fragments, each adapter ligated fragment comprising a first strand and a second strand and an adapter on at least one end of the fragment, wherein at least some of the adapter ligated fragments comprise a repeat element; and
(d) performing amplification using a pair of primers, wherein a first primer of the pair of primers is complementary to a sequence in the first strand of the adapter ligated fragment within the repeat element and the second primer of the pair of primers is complementary to a sequence in the second strand of the adapter ligated fragment within the common hybridization sequence on the adapters, wherein performing the amplification amplifies the nucleic acid between the far ends of the repeat element and the common hybridization sequence on the adapters.

2. The method of claim 1, further comprising creating blunt ends on the double-stranded fragments of genomic nucleic acid prior to adding the double-stranded adapters to the sample.

3. The method of claim 2, further comprising adding a single dA at the 3′ ends of the blunted fragments to create fragments with a 3′ dA overhang on both strands of the fragments.

4. The method of claim 2 or 3, further comprising phosphorylating the 5′ ends of the blunted fragments.

5. The method of claim 4, wherein one end of the adapters comprises a first 3′ dT overhang and 5′ phosphorylated end, and the other end of the adapter is blunted or comprises a second 3′ dT overhang and 5′ phosphorylated end.

6. The method of claim 5, wherein each adapter comprises a unique molecular identifier (UMI) that is between the first or the second dT overhang and the common hybridization sequence and is different from the UMI on any other adapter in the sample.

7. The method of claim 1, wherein the method comprises:
(a) providing a sample containing double-stranded fragments of genomic nucleic acid;
(b) creating blunt ends on the fragments;
(c) adding a single dA at the 3′ ends of the blunted fragments to create fragments with a 3′ dA overhang on both strands of the fragments;
(d) phosphorylating the 5′ ends of the fragments;
(e) adding to the sample double-stranded adapters, wherein one end of the adapter comprises a first 3′ dT overhang and 5′ phosphorylated end, and the other end of the adapter is blunted or comprises a second 3′ dT overhang and 5′ phosphorylated end, and wherein each adapter comprises a common hybridization sequence and a unique molecular identifier (UMI) that is between the first or second dT overhang and the common hybridization sequence and is different from the UMI on any other adapter in the sample;
(f) applying ligation conditions to the sample to form a plurality of double-stranded adapter ligated fragments, each adapter ligated fragment comprising a first strand and a second strand and an adapter on at least one end of the fragment, wherein at least some of the adapter ligated fragments comprise a repeat element; and
(g) performing amplification using a pair of primers, wherein a first primer of the pair of primers is complementary to a sequence in the first strand of the adapter ligated fragment within the repeat element and the second primer of the pair of primers is complementary to a sequence in the second strand of the adapter ligated fragment within the common hybridization sequence on the adapters, wherein performing the amplification amplifies the nucleic acid between the far ends of the repeat element and the common hybridization sequence on the adapters.

8. The method of claim 1, wherein the concentration of the first primer is at least 5 times higher than the concentration of the second primer.

9. The method of claim 1, wherein the amplification comprises PCR or isothermal amplification.

10. The method of claim 9, wherein the amplification comprises PCR, optionally wherein the PCR comprises 2-20 cycles of touch-down PCR followed by COLD-PCR and/or step-up PCR to preferentially amplify repeat elements with a deletion.

11. The method of claim 9 or 10, wherein performing PCR comprises:
extending the first primer that is annealed to the first strand of the adapter ligated fragment within the repeat element and the second primer that is annealed to the second strand of the adapter ligated fragment within the common hybridization sequence on the adapter for a period of time t1 so that the extended primers are 500-600 bp long.

12. The method of claim 11, wherein t1 is 5-60 seconds.

13. The method of any one of claims 1-12, further comprising forming the sample of fragments of genomic nucleic acid from a sample of intact genomic nucleic acid.

14. The method of any one of claims 1-13, wherein the adding to the sample double-stranded adapters and applying ligation conditions results in an increase in the number of repeat elements captured by at least 10-fold compared to a method comprising performing amplification without the adding to the sample double-stranded adapters and applying ligation conditions.

15. The method of any one of claims 1-14, wherein the repeat element is a tandem repeat or a portion thereof, or an interspersed repeat or a portion thereof.

16. The method of claim 15, wherein the tandem repeat is a megasatellite, a minisatellite, or a microsatellite.

17. The method of claim 15, wherein the repeat element is an interspersed repeat that is a Short Interspersed Nuclear Element (SINE) or portion thereof, or a Long Interspersed Nuclear Element (LINE) or a portion thereof.

18. The method of claim 17, wherein the repeat element is a SINE, and the SINE is an Alu element or a portion thereof, wherein the portion thereof is a polyA tail.

19. The method of any one of claims 1-18, wherein the sample of nucleic acid was obtained from a sample of blood comprising less than 1 ng of nucleic acid (or only a few μl).

20. The method of any one of claims 1-19, further comprising:
(a) determining the number of mutations in the amplified regions of genomic nucleic acid, wherein the number of mutations provides an indication of mismatch repair deficiency or total mutation burden;
(b) determining the number of insertions or deletions in homopolymers or heteropolymers in the amplified regions of genomic nucleic acid, wherein the number of insertions or deletions provides an indication of microsatellite instability;
(c) determining the number of copies of a gene of interest in the amplified regions of genomic nucleic acid, wherein the number of copies of the gene of interest provides an indication of disease;
(d) determining the number of methylated forms of a gene of interest in the amplified regions of genomic nucleic acid; or
(e) determining the number of a short tandem repeat in the amplified regions of genomic nucleic acid and comparing to the number of the short tandem repeats in a reference sample.

21. A method of enriching regions of a genome in a sample of genomic nucleic acid, the method comprising:
(a) providing a sample containing double-stranded fragments of genomic nucleic acid;
(b) applying random ligation conditions to the sample to form a plurality of double-stranded concatemers, each double-stranded concatemer having a first repeat element and a second repeat element and each double-stranded concatemer having a first strand and a complementary second strand; and
(c) performing amplification using a pair of primers, wherein a first primer of the pair of primers is complementary to a sequence in the first strand within the first repeat element and the second primer of the pair of primers is complementary to a sequence in the second strand within the second repeat element, wherein performing the amplification amplifies nucleic acid between the first and second repeat elements.

22. The method of claim 21, further comprising blunt ending the double-stranded fragments of genomic nucleic acid before the random ligation of step (b).

23. The method of claim 21, wherein the amplification comprises PCR or isothermal amplification.

24. The method of claim 23, wherein performing PCR comprises:
extending the first primer that is annealed to the first strand of the double-stranded concatemer within the first repeat element and the second primer that is annealed to the second strand of the double-stranded concatemer within the second repeat element for a period of time t1 so that the extended primers are 500-600 bp long.

25. The method of claim 24, wherein t1 is 5-60 seconds.

26. The method of claim 21 or 22, further comprising performing whole-genome amplification on the concatemers before performing the amplification of step (c).

27. The method of any one of the preceding claims, further comprising forming the sample of fragments of genomic nucleic acid from a sample of intact genomic nucleic acid.

28. A method of sequencing regions of a genome, the method comprising sequencing the amplified regions between the first and second repeat elements of any one of the preceding claims.

29. The method of claim 28, further comprising performing single-stranded or double-stranded consensus techniques with unique molecular identifiers (UMI) to identify amplification errors in sequencing data obtained from the amplified regions between the first and second repeat elements on each concatemer, wherein each UMI comprises at least two base pairs of each fragment on either side of a junction between fragments that form the junction.

30. The method of any one of the preceding claims, wherein the applying random ligation conditions results in an increase in amplifiable nucleic acid by at least 2 PCR cycles compared to a method comprising the performing amplification without the applying random ligation conditions.

31. The method of any one of the preceding claims, wherein the first repeat element is a tandem repeat or a portion thereof or an interspersed repeat or a portion thereof and wherein the second repeat element is a tandem repeat or a portion thereof or an interspersed repeat or a portion thereof.

32. The method of claim 31, wherein the tandem repeat is a mega satellite or portion thereof, a minisatellite or portion thereof, or a microsatellite or portion thereof.

33. The method of claim 31, wherein the repeat element is an interspersed repeat that is a Short Interspersed Nuclear Element (SINE) or portion thereof, or a Long Interspersed Nuclear Element (LINE) or a portion thereof.

34. The method of claim 33, wherein the repeat element is a SINE, and the SINE is an Alu element, Alu, or a portion thereof, wherein the portion thereof is a polyA tail.

35. The method of claim 31, wherein the first repeat element is different from the second repeat element.

36. The method of any one of the preceding claims, wherein the applying random ligation conditions comprises blunt end ligation, or single-stranded ligation.

37. The method of any one of the preceding claims, wherein the applying random ligation conditions comprises:
creating blunt ends on the fragments;
adding a single dA at the 3′ ends of the blunted fragments to create fragments with a 3′ dA overhang on both strands of the fragments;
phosphorylating the 5′ ends of the fragments;
adding to the sample double-stranded adapters, wherein each adapter is 4-30 bp long and comprises a 3′ dT and phosphorylated 5′ end on both strands of the adapter and a unique molecular identifier (UMI) such that the UMI on each adapter is different from the UMI on any other adapter in the sample; and
applying ligating conditions to allow ligation between the fragments with the 3′ dA overhangs and the adapters with the 3′ dT overhangs.

38. The method of claim 37, wherein each adapter further comprises 1-2 mismatched bp, wherein the mismatched bp are not the outermost base pairs of the adapter.

39. The method any one of the preceding claims, wherein the sample of nucleic acid was obtained from a sample of blood comprising less than 1 ng of nucleic acid (or only a few μl).

40. The method of any of the preceding claims, further comprising:
(a) determining the number of mutations in the amplified regions of genomic nucleic acid, wherein the number of mutations provides an indication of mismatch repair deficiency or total mutation burden;
(b) determining the number of insertions or deletions in homopolymers or heteropolymers in the amplified regions of genomic nucleic acid, wherein the number of insertions or deletions provides an indication of microsatellite instability;
(c) determining the number of copies of a gene of interest in the amplified regions of genomic nucleic acid, wherein the number of copies of the gene of interest provides an indication of disease;
(d) determining the number of methylated forms of a gene of interest in the amplified regions of genomic nucleic acid; or
(e) determining the number of a short tandem repeat in the amplified regions of genomic nucleic acid and comparing to the number of the short tandem repeat in a reference sample.

41. A method of enriching portions of a genome in a sample of genomic nucleic acid, the method comprising:
(a) providing a sample containing double-stranded fragments of genomic nucleic acid;
(b) creating blunt ends on the fragments;
(c) adding a single dA at the 3′ ends of the blunted fragments to create fragments with a 3′ dA overhang on both strands of the fragments;
(d) phosphorylating the 5′ ends of the fragments;
(e) adding to the sample double-stranded adapters, wherein one end of the adapter comprises a first 3′ dT overhang and 5′ phosphorylated end, and the other end of the adapter is blunted or comprises a second 3′ dT overhang and 5′ phosphorylated end, and wherein each adapter comprises a common hybridization sequence and a unique molecular identifier (UMI) that is between the first or second dT overhang and the common hybridization sequence and is different from the UMI on any other adapter in the sample;
(f) applying ligation conditions to the sample to form a plurality of double-stranded adapter ligated fragments, each adapter ligated fragment comprising a first strand and a second strand and an adapter on at least one end of the fragment, wherein at least some of the adapter ligated fragments comprise a repeat element; and
(g) performing amplification using a pair of primers, wherein a first primer of the pair of primers is complementary to a sequence in the first strand of the adapter ligated fragment within the repeat element and the second primer of the pair of primers is complementary to a sequence in the second strand of the adapter ligated fragment within the common hybridization sequence on the adapters, wherein performing the amplification amplifies the nucleic acid between the far ends of the repeat element and the common hybridization sequence on the adapters.

42. The method of claim 41, wherein the concentration of the first primer is at least 5 times higher than the concentration of the second primer.

43. The method of claim 41, wherein the amplification comprises PCR or isothermal amplification.

44. The method of claim 43, wherein the amplification comprises PCR, optionally wherein the PCR comprises 2-20 cycles of touch-down PCR followed by COLD-PCR and/or step-up PCR to preferentially amplify repeat elements with a deletion.

45. The method of claim 43 or 44, wherein performing PCR comprises:
extending the first primer that is annealed to the first strand of the adapter ligated fragment within the repeat element and the second primer that is annealed to the second strand of the adapter ligated fragment within the common hybridization sequence on the adapter for a period of time t1 so that the extended primers are 500-600 bp long.

46. The method of claim 45, wherein t1 is 5-60 seconds.

47. The method of any one of claims 41-46, further comprising forming the sample of fragments of genomic nucleic acid from a sample of intact genomic nucleic acid.

48. The method of any one of claims 41-47, wherein the adding to the sample double-stranded adapters and applying ligation conditions results in an increase in the number of repeat elements captured by at least 10-fold compared to a method comprising performing amplification without the adding to the sample double-stranded adapters and applying ligation conditions.

49. The method of any one of claims 41-48, wherein the repeat element is a tandem repeat or a portion thereof, or an interspersed repeat or a portion thereof.

50. The method of claim 49, wherein the tandem repeat is a megasatellite, a minisatellite, or a microsatellite.

51. The method of claim 49, wherein the repeat element is an interspersed repeat that is a Short Interspersed Nuclear Element (SINE) or portion thereof, or a Long Interspersed Nuclear Element (LINE) or a portion thereof.

52. The method of claim 51, wherein the repeat element is a SINE, and the SINE is an Alu element or a portion thereof, wherein the portion thereof is a polyA tail.

53. The method any one of claims 41-52, wherein the sample of nucleic acid was obtained from a sample of blood comprising less than 1 ng of nucleic acid (or only a few μl).

54. The method of any one of claims 41-53, further comprising:
(a) determining the number of mutations in the amplified regions of genomic nucleic acid, wherein the number of mutations provides an indication of mismatch repair deficiency or total mutation burden;
(b) determining the number of insertions or deletions in homopolymers or heteropolymers in the amplified regions of genomic nucleic acid, wherein the number of insertions or deletions provides an indication of microsatellite instability;
(c) determining the number of copies of a gene of interest in the amplified regions of genomic nucleic acid, wherein the number of copies of the gene of interest provides an indication of disease;
(d) determining the number of methylated forms of a gene of interest in the amplified regions of genomic nucleic acid; or
(e) determining the number of a short tandem repeat in the amplified regions of genomic nucleic acid and comparing to the number of the short tandem repeats in a reference sample.

55. A method for detecting microsatellites in a tumor sample, the method comprising:
(a) providing a sample containing double-stranded fragments of genomic nucleic acid;
(b) adding to the sample double-stranded adapters, wherein each adapter comprises a common hybridization sequence;
(c) applying ligation conditions to the sample to form a plurality of double-stranded adapter ligated fragments, each adapter ligated fragment comprising a first strand and a second strand and an adapter on at least one end of the fragment, wherein at least some of the adapter ligated fragments comprise a repeat element; and
(d) performing amplification using a pair of primers, wherein a first primer of the pair of primers is complementary to a sequence in the first strand of the adapter ligated fragment within the repeat element and the second primer of the pair of primers is complementary to a sequence in the second strand of the adapter ligated fragment within the common hybridization sequence on the adapters, wherein performing the amplification amplifies the nucleic acid between the far ends of the repeat element and the common hybridization sequence on the adapters.


SEQUENCE LISTING (6 sequences)

SEQ ID NO: 1 - DNA, 15 bp, Artificial Sequence
  Synthetic
acacacacacacaca

SEQ ID NO: 2 - DNA, 18 bp, Artificial Sequence
  Synthetic
cagcagcagcagcagcag

SEQ ID NO: 3 - DNA, 18 bp, Artificial Sequence
  Synthetic
aaaaaaaaaaaaaaaaaa

SEQ ID NO: 4 - DNA, 16 bp, Artificial Sequence
  Synthetic
tttttttttttttttt

SEQ ID NO: 5 - DNA, 11 bp, Artificial Sequence
  Synthetic
aaaaaaaaaaa

SEQ ID NO: 6 - DNA, 11 bp, Artificial Sequence
  Synthetic
ttttttttttt